(19) United States
(12) Patent Application Publication
(10) Pub. No.: US 2008/0311036 A1
(43) Pub. Date: Dec. 18, 2008

(54) Imaging of Neural and Organ Injury or Damage

(75) Inventors: Kevin Ka-Wang Wang, Gainesville, FL (US); Ronald L. Hayes, Gainesville, FL (US); Lewis R. Baxter, JR., Gainesville, FL (US); Laszlo Prokai, Mansfield, TX (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: UNIVERSITY OF FLORIDA RESEARCH FOUNDATION, INC., GAINSVILLE, FL (US)

(21) Appl. No.: 11/914,003

(22) PCT Filed: May 11, 2006
(86) PCT No.: PCT/US2006/018222

§ 371 Date: Jun. 26, 2008

Related U.S. Application Data
(60) Provisional application No. 60/680,282, filed on May 11, 2005.

(51) Int. Cl. A61K 51/08; A61K 51/04

(52) U.S. Cl. 424/1.69; 424/1.65; 424/1.81; 424/1.85

(57) ABSTRACT

In vivo determination of regional damage with neural and organ injury specific imaging agents. Rapid, and non-invasive imaging compositions and methods for assessment of the extent of neurotoxic cell loss or nervous system damage resulting from nervous system injury due to ischemia, stroke, trauma, chemical or electrical insult, acute drug overdose or exposure to substance abuse (such as “recreational drugs”) infection or other insults. The same or similar rapid, and non-invasive imaging compositions and methods for assessment of the extent of the organ injury comprises any damage, injury or infection, functional failure to specific organs such as liver, kidney, prostate, lung, skeletal muscle, heart, pancreas, stomach, small and large intestine, bladder and the reproductive system as well as damage, injury or infection, functional failure to multi-organs, trauma-hemorrhagic shock and sepsis. In particular, neural and organ damage is detected via protease inhibitor-based radionuclide-labeled imaging ligand binding to overactivated proteases (calpains, caspases, cathepsins, proteasome, metalloproteases, granzyme B or other proteases) that are specific to neural or organ injury or damage.

### FIELD OF THE INVENTION

Neural and organ-injury specific compositions and methods for imaging of neural and organ damage.

### BACKGROUND

Acute brain injury can take on different forms. Nerve cells (neurons) in the nervous system including the brain and spinal cord and the peripheral nervous system can become injured. For example nerve cells can be injured following traumatic brain injury (TBI), stroke (ischemic or hemorrhagic), spinal cord injury, electrical discharge-induced injury such as epilepsy, exposure to acute drug overdose or to substance abuse (such as “recreational drugs”) at even a single recreational use of abused substances such “Speed” or methamphetamine (Meth) or chemically related “Ecstasy” or 3,4-methylenedioxymethamphetamine (MDMA). Yet, currently there are no simple, rapid and non-invasive methods to assess the extent of neurotoxic cell loss or brain damage resulting from these conditions in the emergency room and to monitor the long-term neurotoxic impact of such conditions.

Accordingly, the neural pathways of a mammal are particularly at risk if neurons are subjected to mechanical or chemical trauma or to neuropathic degeneration sufficient to put the neurons that define the pathway at risk of dying. A host of neuropathies, some of which affect only a subpopulation or a system of neurons in the peripheral or central nervous systems have been identified to date. The neuropathies, which may affect the neurons themselves or the associated glial cells, may result from cellular metabolic dysfunction, infection, exposure to toxic agents, autoimmunity dysfunction, malnutrition or ischemia. In some cases the cellular dysfunction is thought to induce cell death directly. In other cases, the neuropathy may induce sufficient tissue necrosis to stimulate the body's immune/inflammatory system and the mechanisms of the body's immune response to the initial neural injury then destroys the neurons and the pathway defined by these neurons.

Another common injury to the CNS is stroke, the destruction of brain tissue as a result of intracerebral hemorrhage or infarction. Stroke is a leading cause of death in the developed world. It may be caused by reduced blood flow or ischemia that results in deficient blood supply and death of tissues in one area of the brain (infarction). Causes of ischemic strokes include blood clots that form in the blood vessels in the brain (thrombus) and blood clots or pieces of atherosclerotic plaque or other material that travel to the brain from another location (emboli). Bleeding (hemorrhage) within the brain may also cause symptoms that mimic stroke. The ability to detect such injury is lacking in the prior art.

Mammalian neural pathways also are at risk due to damage caused by neoplastic lesions. Neoplasias of both the neurons and glial cells have been identified. Transformed cells of neural origin generally lose their ability to behave as normal differentiated cells and can destroy neural pathways by loss of function. In addition, the proliferating tumors may induce lesions by distorting normal nerve tissue structure, inhibiting pathways by compressing nerves, inhibiting cerebrospinal fluid or blood supply flow, and/or by stimulating the body's immune response. Metastatic tumors, which are a significant cause of neoplastic lesions in the brain and spinal cord, also similarly may damage neural pathways and induce neuronal cell death.

Protease over-activation is a major theme in traumatic and ischemic and other forms of brain injury. Cells Chemical or physical imbalance appear to trigger overactivation of a number of proteases. These include cysteine proteases (calpain-1 and -2, caspase-3, cathepsin-B and -L [Yamashima, (2000). Prog. Neurobiol. 62, 273-295], metalloproteases (e.g., MMP-2 and -9) [Asahi., Asahi Jung del Fini Lo (2000). J. Cereb. Blood Flow Metab. 20, 1681-1689; Clark, Bou., Chapman Edwards (1997) Neurosci. Lett. 238, 53-56.] and proteasome [Phillips Williams, Adams, Elliott, Tortella (2000). Stroke 31, 1686-1693.]. Of particular interest are calpains and caspases [Wang, K. K. W. (2000). Trends Neurosci. 23, 20-26.]. Calpain is activated during both oncotic (necrotic) and apoptotic cell death in neurons, while caspase-3 is strictly activated in neuronal apoptosis. Evidence demonstrates that both necrotic and apoptotic cell death are present in traumatic and ischemic brain injury. Calpain inhibitors have demonstrated neuroprotective effects against brain injury [Kupina, Nath, Bernath, Inoue, Azuma, Yuen, Wang, and. Hall, (2001) J. Neurotrauma. 18, 1229-1240; Li, Howlett, He, Miyashita, Siddiqui MShuaib. (1998) Neurosci Lett. 1998 May 8; 247(1):17-20; Markgraf, Velayo, Johnson, McCarty, Medhi, Koehl, Chmielewski, Linnik. (1998) Stroke. 1998 January; 29(1):152-8; Kawamura, Nakajima, Ishida, Ohmura, Miura, Takada. Brain Res. 2005 Mar 10; 1037(1-2):59-69], as are caspase inhibitors [Deshmukh. Apoptosis. 1998 December; 3(6):387-94; Cheng, Deshmukh, D'Costa, Demaro, Gidday, Shah, Sun, Jacquin, Johnson, Holtzman. J Clin Invest. 1998 May 1; 101(9):1992-9; Fink, Zhu, Namura, Shimizu-Sasamata, Endres, Ma, Dalkara, Yuan, Moskowitz. J Cereb Blood Flow Metab. 1998 October; 18(10):1071-6; Han, Xu, Choi, Han, Xanthoudakis, Roy, Tam, Vaillancourt, Colucci, Siman, Giroux, Robertson, Zamboni, Nicholson, Holtzman. J Biol Chem. 2002 Aug. 16; 277(33):30128-36.]

As such, a protease inhibitor-based non-invasive imaging method to diagnose, image and monitor various neurological injuries might be feasible

Similar to the nervous system injury, proteases are also overactivated in other organ or multi-organ injury, sepsis, trauma and hemorrhagic shock wherein the organ injury comprises any damage, injury or infection, functional failure to specific organs such as liver, kidney, prostate, lung, skeletal muscle, heart, pancreas, stomach, small and large intestine, bladder and the reproductive system (Sindram et al. Transplantation. 1999 Jul. 15; 68(1):136-40; Wang et al. (2004) J Biomed Sci. 2004 September-October; 11(5):571-8; Canbay A et al. J Pharmacol Exp Ther. 2004 March; 308(3):1191-6; Reid and Belcastro. Am J Respir Crit Care Med. 2000, 162(5):1801-6; Shanely, Zergeroglu, Lennon, Sugiura, Yimlamai, Enns, Belcastro, Powers. Am J Respir Crit Care Med. 2002 Nov. 15; 166(10):1369-74; Arthur, Booker, Belcastro. Can J Physiol Pharmacol. 1999 January; 77(1):42-7; Belcastro Shewchuk, Raj. Mol Cell Biochem. 1998 February; 179(1-2):135-45. Badalamente Hurst Stracher (1995). J Reconstr Microsurg. 1995 November; 11(6):429-37; Shiraishi, Naito and Yoshida. Biol Reprod. 2000 November; 63(5):1538-48; Zhao, Levin, Wein and Levin. Urology. 1997 February; 49(2):293-300; Jani, Ljubanovic, Faubel, Kim, Mischak, Edelstein. Am J Transplant. 2004 August; 4(8):1246-54. Chatterjee, Brown, Cuzzocrea, Zacharowsk, Stewart, Mota-Filipe, McDonald, Thiemermann. Kidney Int. 2001 June; 59(6):2073-83), as well as damage, injury or infection, functional failure to multi-organs. [Ruetten and Thiemermann. Br J Pharmacol. 1997 June; 121(4):695-704; McDonald M C et al. (2001) FASEB J. 2001 January; 15(1):171-186; Cuzzocrea et al. (2002) Crit Care Med. 2002 October; 30(10):2284-94).

As such, a protease inhibitor-based non-invasive imaging method to diagnose, image and monitor various organ or multi-organ injuries is feasible.

These neural or organ injury-specific imaging agent can be detected by positron emission tomography (PET), single photon emission computed tomography (SPECT), radioscintigraphy, magnetic resonance imaging (MRI), computed tomography (CT scan).

### SUMMARY

The compositions described are neural or organ injury-specific imaging agents. In particular, neural and organ damage is detected via proteases and inhibitors that are specific to injury or damage to the central and peripheral nervous system as well as other organs. For example, both TBI and Meth/MDMA exposure differentially trigger the over-activation of two cellular proteases (calpain and caspase) in injured neurons, leading to two different forms of cell death (necrosis-acute cell death and apoptosis-delayed cell death). In neural or organ injury, other protease might also be over-activated, including cathepsins, proteasome, metalloproteinases, Granzyme B and other proteases. Since protease inhibitor binds preferentially to activated protease, thus protease inhibitor-based imaging ligands (e.g. with radionuclide label) can be used as neural or organ injury-specific imaging agents.

In a preferred embodiment, a composition comprising a calpain and/or caspase inhibitor and a radionuclide. The calpain inhibitor comprises calpain inhibitor I, calpain inhibitor II, N-acetyl-Leu-Leu-norleucinal, N-acetyl-Leu-Leu-methioninal, calpeptin, E-64, E-64-c, E-64-d, Z-VF-CHO, Z-Leu-Leu-CHO, leupeptin (N-acetyl-Leu-Leu-argininal), oxoamide inhibitor molecules AK295, AK275, MDL28170 CX275, SJA6017, SNJ-1715 or SNJ-1945.

The caspase inhibitor inhibits activity of caspase-1, caspase-2, caspase-3, caspase-4, caspase-5, caspase-6, caspase-6, caspase-7, caspase-8, or caspase-9. The caspase inhibitor comprises Z-D-DCB (Z-Asp-CH2OC(O)-2,6-dichlorobenzene or zAsp-CH2-DCB), Z-VAD-DCB, zVADfmk, acetyl-DEVD-CHO, DEVD-fluoromethylketone, Z-Val-DL-Asp-fluoromethylketone, Z-Val-DL-Asp(OMe)-fluoromethylketone M826 or IDN-6556.

In a preferred embodiment, cathepsin inhibitor are CA-074, CA-074-Me, CP-1, CP-2, Napsul-Ile-Trp-CHO and Pepstatin A. Examples of granzyme B inhibitor is 3,4-Dichloroisocoumarin.

In another preferred embodiment, metalloproteinase inhibitor comprises Actinonin, CL-82198, Epigallocatechin gallate; GM6001; NNGH (BML-205), BB-94 and KB-R7785. Examples of proteasome inhibitor comprises Lactacystin, Clasto-Lactacystin β-lactone, Epoxomicin, Gliotoxin, MG-132, MG-262, PS-341; Z-Ile-Glu(OtBu)-Ala-Leu-CHO (IGAL) and MN-519; examples of other protease inhibitor includes, but not limited to AAF-CMK, Arphamenine A, Bestatin (Ubenimex), Boc-GVV-CHO; Captopril, Elastatinal, Phosphoramidon, PPACK, Z-Prolyl-Prolinal, Thiorphan (DL), TLCK and TPCK.

In another preferred embodiment, the radionuclide comprises ¹¹C, ¹⁴C, ³H, ¹⁸F, ^{99m}T, ¹⁸⁶Re, ¹⁸⁸Re, ¹¹¹In, ⁶⁷Ga, ⁶⁸Ga, ²⁰¹Tl, ⁵²Fe, ²⁰³Pb, ⁵⁸Co, ⁶⁴Cu, ¹²³I, ¹²⁴I, ¹²⁵I, ¹³¹I, ²¹⁰At, ⁷⁶Br, ⁷⁷Br, ¹⁸F, ³²P, ³⁵S, ¹²³I, ¹²⁵I, or ¹³¹I. Preferably, the radioactive ligand is ¹¹C, ¹⁴C or ¹⁸F. The calpain and/or caspase inhibitor is preferably ligated to the radionuclide via a covalent bond.

In another preferred embodiment, a method of neural/organ-injury imaging comprises administering to a patient a neural specific imaging agent; detecting the radiolabeled calpain and/or caspase inhibitor; thereby imaging neural damage in the patient. The neural damage comprises any one of: damage to retinal ganglion cells; a traumatic brain injury; a stroke related injury; a cerebral aneurism related injury; demyelinating diseases; a spinal cord injury, including monoplegia, diplegia, paraplegia, hemiplegia and quadriplegia; a neuroproliferative disorder or neuropathic pain syndrome; stroke, concussion, post-concussion syndrome, cerebral ischemia, neurodegenerative diseases brain injuries, or neuropathies.

In another preferred embodiment, the neural/organ injury specific imaging agent is a calpain and/or caspase inhibitor. For example, SNJ-1715 (Masayuki Nakamura, Masazumi Yamaguchi, Osamu Sakai, Jun Inoue Bioorganic & Medicinal Chemistry 11, 1371-1379 (2003)). Calpastatin is an endogenous inhibitor of most calpains, the tissue-specific calpain p94 being an exception. Calpastatin, which has five domains, is cleaved by calpain in the interdomain regions, generating inhibitory peptides. The inhibitory effect of calpastatin has been attributed to interactions with calpain domains II, III, IV, and VI. The reactive site of calpastatin shows no apparent homology to that of other protease inhibitors, and it contains the consensus sequence TIPPXYR (SEQ ID NO:1), which is essential for inhibition. Synthetic active-site inhibitors with varying specificities for calpain and other cysteine proteases include E-64 and derivatives of E-64; leupeptin (N-acetyl-Leu-Leu-argininal); calpain inhibitors I (N-acetyl-Leu-Leu-norleucinal) and II (N-acetyl-Leu-Leu-methioninal); oxoamide inhibitor molecules AK295, AK275, and CX275; and derivatives of peptidyl α-oxo compounds. In contrast to these active-site inhibitors, PD150606 inhibit calpains by binding the calcium-binding domains. The combination of PD150606 and an active site inhibitor such as AK295 can inhibit calpain with high specificity.

In a preferred embodiment, several typical tissue-specific calpains in vertebrates, include skeletal muscle p94 (nCL-1, calpain 3′, CAPN3), stomach nCL2 (CAPN4) and nCL 2′, and digestive tubule nCL4. While p94 contains EF hands, it does not require calcium for proteinase activity. p94 has a domain IV sequence similar to that of μCL and mCL, but it does not bind to a small 30 kDa subunit. p94 contains unique insertion sequences called IS1 and IS2, which are found in domain II and between domains III and IV, respectively). IS2 contains a nuclear-localization-signal-like basic sequence (Arg-Pro-Xaa-Lys-Lys-Lys-Lys-x-Lys-Pro). Connectin/titin binding is also attributed to IS2. p94 may change its localization in a cell-cycle dependent manner and may be involved in muscle differentiation by interacting with the MyoD family.

In a preferred embodiment, biopeptides indicative of in vivo injury can include proteolytic peptides, calpain substrates, calpain, activators of calpain. For example, calpain substrates include “PEST” proteins, which have high proline, glutamine, serine, and threonine contents; calpain and calpastatin; signal transduction proteins including protein kinase C, transcription factors c-Jun, c-Fos, and α-subunit of heterotrimeric G proteins; proteins involved in cell proliferation and cancer including p53 tumor suppressor, growth factor receptors (e.g., epidermal growth factor receptor), c-Jun, c-Fos, and N-myc; proteins with established physiological roles in muscle including Ca²⁺-ATPase, Band III, troponin, tropomyosin, and myosin light chain kinase; myotonin protein kinase; proteins with established physiological roles in the brain and the central nervous system including myelin proteins, myelin basic protein (MBP), axonal neurofilament protein (NFP), myelin protein MAG; cytoskeletal and cell adhesion proteins including troponins, talin, neurofilaments, spectrin, microtubule associated protein MAP-2, tau, MAPIB, fodrin, desmin, α-actinin, vimentin, spectrin, integrin, cadherin, filamin, and N-CAM; enzymes including protein kinases A and C, and phospholipase C; and histones. Illustrative examples are also listed in Table 1.

In a preferred embodiment, selective inhibitors of calpain, for neural/organ injury specific imaging, are identified by the substrate specificity of calpain. For example, a selective calpain inhibitor is determined based on classes of substrates and specific substrates. Substrates of calpain have been associated into several classes including cytoskeletal and structural proteins, membrane bound receptors and proteins, calmodulin binding proteins, enzymes myofibrillar proteins and transcription factors. The examples of the first group include spectrin, MAP-2a, tau factor, neurofilament H, M and L, α-actinin. Examples of the second class include EGF receptor, AMPA-receptor, calcium pump, anion channel, calcium release channel, L-type calcium channel, G-proteins. Examples of the third class include calcium pump, calcineurin, CaM-dependent protein kinase II, myosin light chain kinase, neuromodulin, connexins, IP3 kinase. Examples of the fourth group include protein kinase C, HMG-CoA reductase, cAMP-dependent kinase, pyruvate kinase, phosphorylase kinase. Examples of the fifth group include troponin I, troponin T, tropomyosin, myosin. Examples of the sixth group include c-fos, c-jun, Pit-1, Oct-1, and b, c-Myc. See Wang, et al. (1997) Advances in Pharmacology, Volume 37.

In another preferred embodiment, the neural/organ injury specific imaging agent further comprises a radionuclide. The radionuclide preferably comprises any one of: ¹¹C, ¹⁴C, ³H, ¹⁸F, ^{99m}T, ¹⁸⁶Re, ¹⁸⁸Re, ¹¹¹In, ⁶⁷Ga, ⁶⁸Ga ²⁰¹Tl, ⁵²Fe, ²⁰³Pb, ⁵⁸Co, ⁶⁴Cu, ¹²³I, ¹²⁴I, ¹²⁵I, ¹³¹I, ²¹⁰At, ⁷⁶Br, ⁷⁷Br, ¹⁸F, ³²P, ³⁵S, ¹²³I, ¹²⁵I, or ¹³¹I.

In another preferred embodiment, the neural/organ injury specific imaging agent is detected by positron emission tomography (PET), single photon emission computed tomography (SPECT), radioscintigraphy, magnetic resonance imaging (MRI), computed tomography (CT scan).

In another preferred embodiment, a method of diagnosing neural damage in a patient, comprises administering to a patient a neural/organ injury specific imaging agent; detecting the radiolabeled calpain and/or caspase inhibitor; thereby, diagnosing neural damage in the patient.

The neural/organ injury specific imaging agent comprises a calpain and/or caspase inhibitor ligated to a radionuclide, such as, for example, ¹¹C, ¹⁴C, ³H, ¹⁸F, ^{99m}T, ¹⁸⁶Re, ¹⁸⁸Re, ¹¹¹In, ⁶⁷Ga, ⁶⁸Ga, ²⁰¹Tl, ⁵²Fe, ²⁰³Pb, ⁵⁸Co, ⁶⁴Cu, ¹²³I, ¹²⁴I, ¹²⁵I, ¹³¹I, ²¹⁰At, ⁷⁶Br, ⁷⁷Br, ¹⁸F, ³²P, ³⁵S, ¹²³I, ¹²⁵I, or ¹³¹I.

In accordance with the invention, the neural/organ injury specific imaging agent is detected by positron emission tomography (PET), single photon emission computed tomography (SPECT), radioscintigraphy, magnetic resonance imaging (MRI), computed tomography (CT scan).

In another preferred embodiment, a kit comprises a calpain and/or caspase inhibitor and a radionuclide; wherein, the calpain inhibitor comprises calpain inhibitor I, calpain inhibitor II, N-acetyl-Leu-Leu-norleucinal, N-acetyl-Leu-Leu-methioninal, calpeptin, E-64, E-64-c, E-64-d, Z-VF-CHO, Z-Leu-Leu-CHO, leupeptin (N-acetyl-Leu-Leu-argininal), oxoamide inhibitor molecules AK295, AK275, MDL28170 CX75, SJA6017, SNJJ-1715 or SNJ-1945; and, the caspase inhibitor comprises Z-D-DCB (Z-Asp-CH2OC(O)-2,6-dichlorobenzene or zAsp-CH2-DCB), Z-VAD-DCB, zVADfmk, acetyl-DEVD-CHO, DEVD-fluoromethylketone, Z-Val-DL-Asp-fluoromethylketone, Z-Val-DL-Asp(OMe)-fluoromethylketone M826 or IDN-6556. Preferably the radionuclide comprises ¹¹C, ¹⁴C, ³H, ¹⁸F, ^{99m}T, ¹⁸⁶Re, ¹⁸⁸Re, ¹¹¹In, ⁶⁷Ga, ⁶⁸Ga, ²⁰¹Tl, ⁵²Fe, ²⁰³Pb, ⁵⁸Co, ⁶⁴Cu, ¹²³I, ¹²⁴I, ¹²⁵I, ¹³¹I, ²¹⁰At, ⁷⁶Br, ⁷⁷Br, ¹⁸F, ³²P, ³⁵S, ¹²³I, ¹²⁵I, or ¹³¹I.

Other aspects of the invention are discussed infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a schematic illustration showing that intracellular proteases calpains and caspases are activated acutely and in a delayed fashion, respectively in nervous system injury such as TBI and METH exposure. Protease inhibitor based SPECT and PET ligands can be used to track the injured brain cells by live imaging. Similarly, other organ injuries also trigger protease activation and these protease inhibitor based SPECT and PET ligands can be used to image organ injury

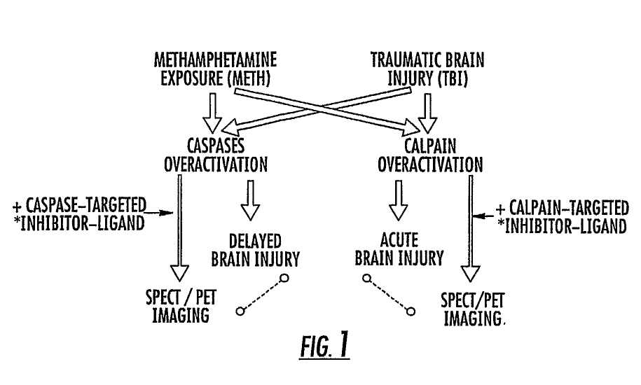

FIG. 2 shows examples of calpain inhibitors that can be modified into neural or organ injury imaging ligands.

FIG. 3 shows examples of caspase inhibitors that can be modified into neural or organ injury imaging ligands

FIG. 4 shows examples of several brain penetrating calpain inhibitors and caspase inhibitors of which ¹²⁵I-label, ¹⁴C-label or other radioisotope can be incorporated into.

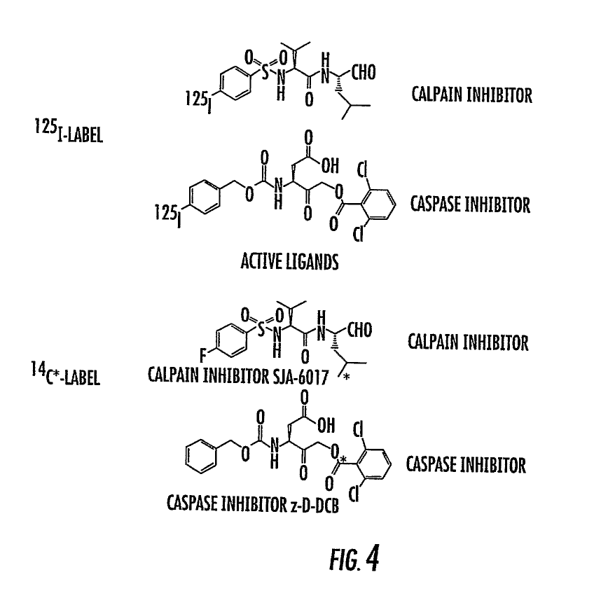

FIG. 5 is a schematic illustration showing examples of several brain penetrating calpain and caspase inhibitors that can be synthesized as blood-brain barrier (BBB)-permeable pro-drugs for calpain inhibitor (based on SJA6017) and caspase inhibitor (based on Z-Asp-DCB) to enhance BBB-permeability and brain residency, and ¹²⁵I-label, ¹⁴C-label (or other radioisotope) can be incorporated into these compounds.

### DETAILED DESCRIPTION

A novel neural and organ-injury specific compositions and methods for imaging of neural and organ damage is described. Rapid, and non-invasive imaging compositions and methods for assessment of the extent of neurotoxic cell loss or nervous system damage resulting from nervous system injury due to ischemia, stroke, trauma (e.g. TBI), chemical or electrical insult, acute drug overdose or exposure to substance abuse (such as “recreational drugs” Meth/MDMA) infection or other insults. The same or similar rapid, and non-invasive imaging compositions and methods for assessment of the extent of the organ injury comprises any damage, injury or infection, functional failure to specific organs such as liver, kidney, prostate, lung, skeletal muscle, heart, pancreas, stomach, small and large intestine, bladder and the reproductive system as well as damage, injury or infection, functional failure to multi-organs, trauma-hemorrhagic shock and sepsis. In particular, neural and organ damage is detected via protease inhibitor-based radionuclide-labeled imaging ligand binding to overactivated proteases (calpains, caspases, cathepsins, proteasome, metalloproteases, Granzyme B or other proteases) that are specific to neural or organ injury or damage.

Similar to the nervous system, proteases are also overactivated in other organ or multi-organ injury, sepsis, trauma and hemorrhagic shock wherein the organ injury comprises any damage, injury or infection, functional failure to specific organs such as liver, kidney, prostate, lung, skeletal muscle, heart, pancreas, stomach, small and large intestine, bladder and the reproductive system

These neural or organ injury-specific imaging agent can be detected by positron emission tomography (PET), single photon emission computed tomography (SPECT), radioscintigraphy, magnetic resonance imaging (MRI), computed tomography (CT scan). For example, our data showed that both TBI and Meth/MDMA exposure differentially trigger the over-activation of two cellular proteases (calpain and caspase) in injured neurons, leading to two different forms of cell death (necrosis and apoptosis). Since both proteases are only activated during pathological events, they are ideal targets for positron emission tomography (PET)-based imaging of brain injury. A PET-visible radioactive isotope (such as ¹⁸F or ¹¹C) can be incorporated into brain-penetrating calpain and caspase inhibitors to serve as “inhibitor-ligands”. The specificity and potency of the calpain and caspase inhibitor-ligands are first validated with labeling of their respective proteases in neural cells in culture and subsequently in injured brain sections. These “inhibitor-ligands” are then further validated in vivo by introducing them through the blood stream into animals after TBI or Meth exposure to allow for micro PET-based live imaging of injured neurons.

These novel neural or organ injury-specific, PET-visible inhibitor-ligands can be readily incorporated into existing micro PET and PET platform technology and broadly used for monitoring acute brain injury progression, response to drug treatment, provides an attending physician with, for example, diagnoses of neural anomalies, assist the physician in making a diagnosis, or assessing the need for, or likely success of, surgery, or can be employed simply to confirm or question the physician's diagnoses.

### DEFINITIONS

Prior to setting forth the invention, it may be helpful to an understanding thereof to set forth definitions of certain terms that will be used hereinafter.

“Activity” of an enzyme is the amount of product produced per unit time at a fixed temperature and pH.

“Specific activity” of an enzyme is the amount of product produced per unit time per mg protein.

“Substrate” is the target protein that the enzyme catalyzes. The International Union of Biochemistry (I.U.B.) initiated standards of enzyme nomenclature which recommend that enzyme names indicate both the substrate acted upon and the type of reaction catalyzed. For example, under this system, the enzyme uricase is called urate: O₂ oxidoreductase, while the enzyme glutamic oxaloacetic transaminase (GOT) is called L-aspartate: 2-oxoglutarate aminotransferase.

As used herein, “inhibitors” refers to any molecule that inhibits the activity of any enzyme that is indicative of neural damage. Examples of the desired target families are shown in Table 1.

As used herein, “inhibitory concentration” is intended to mean the concentration at which the “potential inhibitor of calpain, caspase” compounds screened in the enzyme assays inhibit a measurable percentage of calpain, caspases or any protein shown in Table 1. Examples of “inhibitory concentration” values range from IC₅₀ to IC₉₀, and are preferably, IC₅₀, IC₆₀, IC₇₀, IC₈₀, or IC₉₀, which represent 50%, 60%, 70%, 80% and 90% reduction in calpain or caspase mediated damage. More preferably, the “inhibitory concentration” is measured as the IC₅₀ value. It is understood that an designation for IC₅₀ is the half maximal inhibitory concentration.

As used herein “calpain mediated physiological damage” refers to pathological conditions mediated by calpain. Such conditions can include a variety of ischemic events (such as myocardial or cerebral ischemia), as well as non-ischemic disorders (such as Alzheimer's disease or muscular dystrophy).

“Neural cells” as defined herein, are cells that reside in the brain, central and peripheral nerve systems, including, but not limited to, nerve cells, glial cell, oligodendrocyte, microglia cells or neural stem cells.

“Neural/organ injury specific or neuronally enriched proteins” are defined herein, as proteins that are present in neural cells and not in non-neuronal cells, such as, for example, cardiomyocytes, myocytes, in skeletal muscles, hepatocytes, kidney cells and cells in testis.

“Neural (neuronal) defects, disorders, neuropathies or diseases” as used herein refers to any neurological disorder, including but not limited to neurodegenerative disorders (Parkinson's; Alzheimer's) or autoimmune disorders (multiple sclerosis) of the central nervous system; memory loss; long term and short term memory disorders; learning disorders; autism, depression, benign forgetfulness, childhood learning disorders, close head injury, and attention deficit disorder; autoimmune disorders of the brain, neuronal reaction to viral infection; brain damage; depression; psychiatric disorders such as bi-polarism, schizophrenia; narcolepsy/sleep disorders (including circadian rhythm disorders, insomnia and narcolepsy); severance of nerves or nerve damage; severance of the cerebrospinal nerve cord (CNS) and any damage to brain or nerve cells; neurological deficits associated with AIDS; tics (e.g. Giles de la Tourette's syndrome); Huntington's chorea, schizophrenia, traumatic brain injury, tinnitus, neuralgia, especially trigeminal neuralgia, neuropathic pain, inappropriate neuronal activity resulting in neurodysthesias in diseases such as diabetes, MS and motor neuron disease, ataxias, muscular rigidity (spasticity) and temporomandibular joint dysfunction; Reward Deficiency Syndrome (RDS) behaviors in a subject.

As used herein, the term “injury or neural injury” is intended to include a damage which directly or indirectly affects the normal functioning of the CNS or PNS. For example, the injury can be damage to retinal ganglion cells; a traumatic brain injury; a stroke related injury; a cerebral aneurism related injury; demyelinating diseases such as multiple sclerosis; a spinal cord injury, including monoplegia, diplegia, paraplegia, hemiplegia and quadriplegia; a neuroproliferative disorder or neuropathic pain syndrome. Examples of CNS injuries or disease include TBI, stroke, concussion (including post-concussion syndrome), cerebral ischemia, neurodegenerative diseases of the brain such as Parkinson's disease, Dementia Pugilistica, Huntington's disease and Alzheimer's disease, brain injuries secondary to seizures which are induced by radiation, exposure to ionizing or iron plasma, nerve agents, cyanide, toxic concentrations of oxygen, neurotoxicity due to CNS malaria or treatment with anti-malaria agents, malaria pathogens, injury due to trypanosomes, and other CNS traumas. Examples of PNS injuries or diseases include neuropathies induced either by toxins (e.g. cancer chemotherapeutic agents) diabetes, peripheral trauma or any process that produced pathological destruction of peripheral nerves and/or their myclin sheaths.

As used herein, the term “stroke” is art recognized and is intended to include sudden diminution or loss of consciousness, sensation, and voluntary motion caused by rapture or obstruction (e.g. by a blood clot) of an artery of the brain.

As used herein, the term “Traumatic Brain Injury” is art recognized and is intended to include the condition in which, a traumatic blow to the head causes damage to the brain, often without penetrating the skull. Usually, the initial trauma can result in expanding hematoma, subarachnoid hemorrhage, cerebral edema, raised intracranial pressure (ICP), and cerebral hypoxia, which can, in turn, lead to severe secondary events due to low cerebral blood flow (CBF).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, benzenesulfonic, toluenesulfonic, methanesulfonic, ethane disulfonic, oxalic, isethionic, and the like. Lists of suitable salts are found in Remington's Pharmaceutical Sciences, 17th ed., Mack Publishing Company, Easton, Pa., 1985, p. 1418, the disclosure of which is hereby incorporated by reference.

The term “metallopharmaceutical” means a pharmaceutical comprising a metal. The metal is the cause of the imagable signal in diagnostic applications and the source of the cytotoxic radiation in radiotherapeutic applications. Radiopharmaceuticals are metallopharmaceuticals in which the metal is a radioisotope.

By “reagent” is meant a compound of this invention capable of direct transformation into a metallopharmaceutical of this invention. Reagents may be utilized directly for the preparation of the metallopharmaceuticals of this invention or may be a component in a kit of this invention.

A “reducing agent” is a compound that reacts with a radionuclide, which is typically obtained as a relatively unreactive, high oxidation state compound, to lower its oxidation state by transferring electron(s) to the radionuclide, thereby making it more reactive.

“Ancillary” or “co-ligands” are ligands that are incorporated into a radiopharmaceutical during its synthesis. They serve to complete the coordination sphere of the radionuclide together with the chelator or radionuclide bonding unit of the reagent. For radiopharmaceuticals comprised of a binary ligand system, the radionuclide coordination sphere is composed of one or more chelators or bonding units from one or more reagents and one or more ancillary or co-ligands, provided that there are a total of two types of ligands, chelators or bonding units. For example, a radiopharmaceutical comprised of one chelator or bonding unit from one reagent and two of the same ancillary or co-ligands and a radiopharmaceutical comprised of two chelators or bonding units from one or two reagents and one ancillary or co-ligand are both considered to be comprised of binary ligand systems. For radiopharmaceuticals comprised of a ternary ligand system, the radionuclide coordination sphere is composed of one or more chelators or bonding units from one or more reagents and one or more of two different types of ancillary or co-ligands, provided that there are a total of three types of ligands, chelators or bonding units. For example, a radiopharmaceutical comprised of one chelator or bonding unit from one reagent and two different ancillary or co-ligands is considered to be comprised of a ternary ligand system.

Ancillary or co-ligands useful in the preparation of radiopharmaceuticals and in diagnostic kits useful for the preparation of said radiopharmaceuticals can comprise one or more oxygen, nitrogen, carbon, sulfur, phosphorus, arsenic, selenium, and tellurium donor atoms. A ligand can be a transfer ligand in the synthesis of a radiopharmaceutical and also serve as an ancillary or co-ligand in another radiopharmaceutical. Whether a ligand is termed a transfer or ancillary or co-ligand depends on whether the ligand remains in the radionuclide coordination sphere in the radiopharmaceutical, which is determined by the coordination chemistry of the radionuclide and the chelator or bonding unit of the reagent or reagents.

A “chelator” or “bonding unit” is the moiety or group on a reagent that binds to a metal ion through the formation of chemical bonds with one or more donor atoms.

### Neural/Organ Injury Specific Imaging Agents

For the preparation of the radiolabeled complex as described herein, four amino acids (the radiolabeling moiety) are covalently linked (or complexed) to a selected radionuclide.

In a preferred embodiment, Carbon-11 (¹¹C) and carbon-14 (¹⁴C) are selected as the radionuclide. is selected as the radionuclide. However, examples of other suitable radionuclides which can be complexed with this moiety include, but are not limited to: ¹¹C, ¹⁴C, ³H, ¹⁸F, ^{99m}T, ¹⁸⁶Re, ¹⁸⁸Re, ¹¹¹In, ⁶⁷Ga, ⁶⁸Ga, ²⁰¹Tl, ⁵²Fe, ²⁰³Pb, ⁵⁸Co, ⁶⁴Cu, ¹²³I, ¹²⁴I, ¹²⁵I, ¹³¹I, ²¹⁰At, ⁷⁶Br, ⁷⁷Br, ¹⁸F, ³²P, ³⁵S, ¹²³I, ¹²⁵I, ¹³¹I.

In addition, similar technology can be applied to the Single-Photon Emission Computed Tomography (SPECT), which requires less expensive equipment set-up. In a preferred embodiment, selective inhibitors of calpain are identified by the substrate specificity of calpain. For example, a selective calpain inhibitor is determined based on classes of substrates and specific substrates. Substrates of calpain have been associated into several classes including cytoskeletal and structural proteins, membrane bound receptors and proteins, calmodulin binding proteins, enzymes myofibrillar proteins and transcription factors. The examples of the first group include spectrin, MAP-2a, tau factor, neurofilament H, M and L, α-actinin. Examples of the second class include EGF receptor, AMPA-receptor, calcium pump, anion channel, calcium release channel, L-type calcium channel, G-proteins. Examples of the third class include calcium pump, calcineurin, CaM-dependent protein kinase II, myosin light chain kinase, neuromodulin, connexins, IP3 kinase. Examples of the fourth group include protein kinase C, HMG-CoA reductase, cAMP-dependent kinase, pyruvate kinase, phosphorylase kinase. Examples of the fifth group include troponin I, troponin T, tropomyosin, myosin. Examples of the sixth group include c-fos, c-jun, Pit-1, Oct-1, and b, c-Myc. See Wang, et al. (1997) Advances in Pharmacology, Volume 37.

In a preferred embodiment, calpain and caspase inhibitors are radioactively labeled The calpain and caspase and/or calpain and caspase inhibitors are activated during pathological events and are ideal targets for positron emission tomography (PET) and other imaging technology such as Single-Photon Emission Computed Tomography (SPECT).

Suitable radioisotopes are known to those skilled in the art and include, for example, isotopes of halogens (such as chlorine, fluorine, bromine and iodine), and metals including technetium and indium. Preferred radioisotopes include ¹¹C, ¹⁴C, ³H, ¹⁸F, ^{99m}T, ¹⁸⁶Re, ¹⁸⁸Re, ¹¹¹In, ⁶⁷Ga, ⁶⁸Ga, ²⁰¹Tl, ⁵²Fe, ²⁰³Pb, ⁵⁸Co, ⁶⁴Cu, ¹²³I, ¹²⁴I, ¹²⁵I, ¹³¹I, ²¹⁰At, ⁷⁶Br, ⁷⁷Br, ¹⁸F, ³²P, ³⁵S, ¹²³I, ¹²⁵I, ¹³¹I.

In another preferred embodiment, the inhibitor compounds include inhibitors of calpains, caspases, cathepsin, Granzyme B, matrix metalloproteases, proteosomes and other proteases. Examples of protease inhibitors include but not limited to: SNJ-1715 (inhibitor of calpain); Z-D-DCB (inhibitor of caspase); CA074, CP-1 (inhibitors of cathepsin B/L); BB-94, KB-R7785 (inhibitors of MMP-2/9); Z-Ile-Glu(OtBu)-Ala-Leu-CHO (IGAL), MN519 (proteasome inhibitors). SNJ-1715 inhibits calpain mediated oncosis, apoptosis and inflammation; Z-D-DCB inhibits caspase mediated apoptosis and inflammation; CA074 and CP-1 inhibit cathepsin B/L mediated excessive cell protein turnover, ECM degradation, and autophagic cell death; BB-94, KB-R7785 inhibit MMP-2/9 mediated ECM destruction; IGAL, MN519-inhibit proteasome mediated cellular protein turnover. Examples of inhibitors in each group are listed in Table 1.

TABLE 1

| TARGET | | |
|---|---|---|
| FAMILY | INHIBITOR | TARGET |
| Calpains | Calpain Inhibitor I | Calpain |
| | (ALLN) | |
| | Calpain Inhibitor II | Calpain; Cathepsins L and B |
| | (ALLM) | |
| | Calpeptin | Calpain; Cathepsin L |
| | E-64 | Calpain; Papain; Cathepsins |
| | E-64-c | Calpain; Papain; Cathepsins |
| | E-64-d | Calpain; Cathepsins |
| | MDL-28170 (Z-VF-CHO) | Calpain; Cathepsin B |
| | Z-Leu-Leu-CHO | Calpain |
| | AK275, AK295 | Calpain |
| | Leupeptin | Calpain |
| | SJA6017 | Calpain |
| | SNJ-1945 | Calpain |
| | BDA-410 (Mitsubishi | Calpain |
| | WO-9801130) | |
| Caspases | Ac-YVAD-CHO | Caspase-1 |
| | Cell-Permeable | Caspase-1 |
| | YVAD-CHO | |
| | Biotin-YVAD-CMK | Caspase-1 |
| | Ac-DEVD-CHO | Caspase-3 |
| | Biotin-DEVD-CHO | Caspase-3 |
| | Cell-Permeable | Caspase-3 |
| | DEVD-CHO | |
| | Ac-IETD-CHO | Caspases-6, -8 |
| | Ac-VDVAD-CHO | Caspase-2 |
| | Casputin ™Reagent | Caspases-3, -7 |
| | Ac-DQMD-CHO | Caspase-3 |
| | Ac-LEHD-CHO | Caspase-9 |
| | Ac-VEID-CHO | Caspase-6 |
| | Z-VAD-FMK, methyl | Caspases |
| | ester | |
| | Z-Asp-CH₂-DCB | Caspases |
| | Caspase-3 Antisense | Caspase-3 |
| | Oligo Pair | |
| | Caspase-7 Antisense | Caspase-7 |
| | Oligo Pair | |
| | M826 | Caspase-3, 7 |
| | IDN-6556 | Caspase-3, 7 |
| Cathepsins | CA-074 | Cathepsin B |
| | CA-074-Me | Cathepsin B |
| | Leupeptin | Cathepsin B; Trypsin; |
| | | Plasmin; Papain |
| | Napsul-Ile-Trp-CHO | Cathepsin L |
| | Pepstatin A | Cathepsin D; Pepsin; Renin |
| | CP-1, CP-2 | Cathepsin B, L |
| Granzyme B | 3,4-Dichloro- | Granzyme B; Serine proteases |
| | isocoumarin | |
| Matrix | Actinonin | MMP-1,3,7,8,9,12; |
| Metallo- | | Aminopeptidases |
| proteinases | CL-82198 | MMP-13 |
| | Epigallocatechin | MMP-2,9,12 |
| | gallate | |
| | GM6001 | MMP-1,2,3,7,8,9,12,14,17 |
| | NNGH (BML-205) | MMP-1,3,7,12 |
| | Phosphoramidon | MMPs, |
| | BB-94 | |
| | KB-R7785 | |
| Proteasome | Lactacystin | Proteasome |
| | Clasto-Lactacystin | Proteasome |
| | β-lactone | |
| | Epoxomicin | Proteasome |
| | Gliotoxin | Proteasome |
| | MG-132 | Proteasome |
| | MG-262 | Proteasome |
| | PS-341 | Proteasome |
| | MN519 | Proteasome |
| | IGAL | Proteasome |
| Other | AAF-CMK | Tripeptidyl peptidase II |
| Proteases | Arphamenine A | Aminopeptidase B |
| (including | Bestatin (Ubenimex) | Aminopeptidases |
| serine | Boc-GVV-CHO | γ-secretase |
| proteases) | Captopril | Angiotensin-converting |
| | | enzyme |
| | Elastatinal | Neutrophil elastase; |
| | | Rhinovirus 2A proteinase |
| | Phosphoramidon | Neutral endopeptidase 24.11; |
| | | Endothelin-converting enzyme |
| | PPACK | Thrombin |
| | Z-Prolyl-Prolinal | Prolyl endopeptidase |
| | Thiorphan (DL) | Neural endopeptidase 24.11 |
| | TLCK | Trypsin-like serine proteases |
| | TPCK | Chymotrypsin-like serine |
| | | proteases |

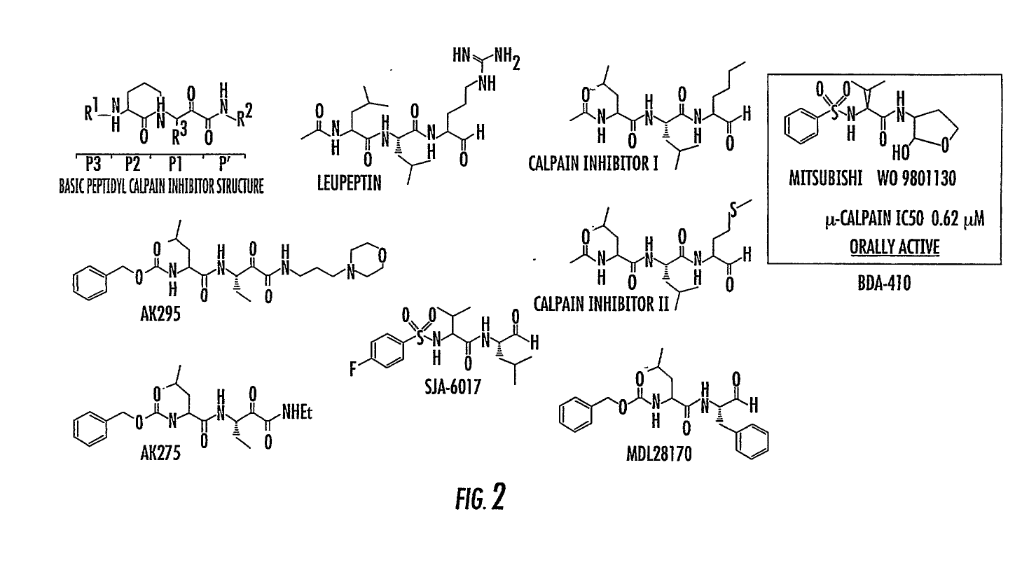

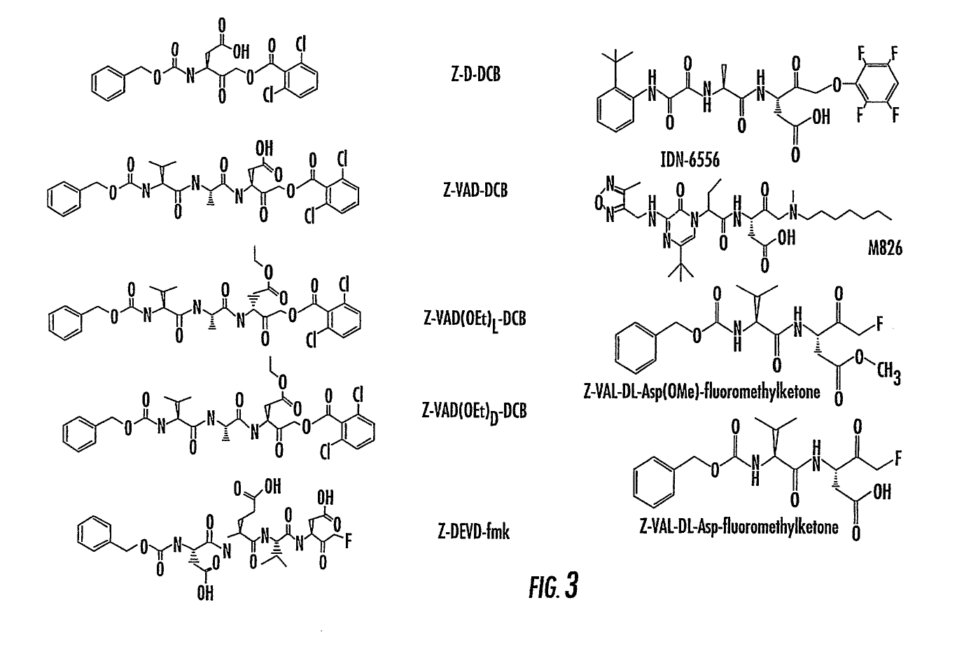

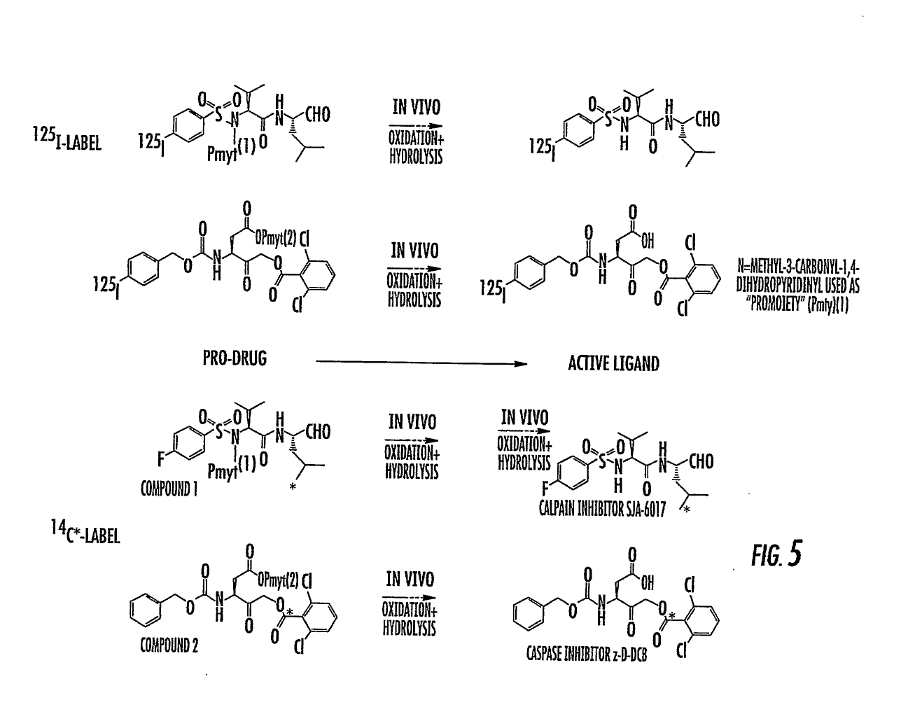

Radiolabeled compounds of the invention may be prepared using standard radiolabeling procedures well known to those skilled in the art. The calpain, caspase and/or calpain and caspase inhibitor compounds of the invention may be radiolabeled either directly (that is, by incorporating the radiolabel directly into the compounds) or indirectly (that is, by incorporating the radiolabel into the compounds through a chelating agent, where the chelating agent has been incorporated into the compounds) (see FIGS. 2 and 3). Also, the radiolabeling may be isotopic or nonisotopic. With isotopic radiolabeling, one group already present in the compounds of the invention described above is substituted with (exchanged for) the radioisotope. With nonisotopic radiolabeling, the radioisotope is added to the compounds without substituting with (exchanging for) an already existing group. Direct and indirect radiolabeled compounds, as well as isotopic and nonisotopic radiolabeled compounds are included within the phrase “radiolabeled compounds” as used in connection with the present invention. Such radiolabeling should also be reasonably stable, both chemically and metabolically, applying recognized standards in the art. Also, although the compounds of the invention may be labeled in a variety of fashions with a variety of different radioisotopes, as those skilled in the art will recognize, such radiolabeling should be carried out in a manner such that the high binding affinity and specificity of the unlabeled or untagged inhibitors of calpain and caspase compounds of the invention to the macromolecule involved in processing is not significantly affected. By not significantly affected, it is meant that the binding affinity and specificity is not affected more than about 3 log units, preferably not more than about 2 log units, more preferably not more than about 1 log unit, even more preferably not more than about 500%, and still even more preferably not more than about 250%, and most preferably the binding affinity and specificity is not affected at all. Radiolabel calpain and caspase inhibitor-imaging ligands can be further improved by being synthesized as blood-brain barrier (BBB)-permeable pro-drugs for calpain inhibitor (based on SJA6017) and caspase inhibitor (based on Z-Asp-DCB) to enhance BBB-permeability and brain residency, and ¹²⁵I-label, ¹⁴C or other radioisotope can be incorporated into these compounds (see FIG. 5).

Selection of a suitable isotope for PET imaging is a difficult task. In general, it is highly desirable the isotope does not have decays other than 511-keV positron emission. This will minimize the impairment of the spatial resolution due to energy and will reduce the radiation burden to the patient. A generator-based isotope is needed due to the high specific activity for receptor-based target specific radiopharmaceuticals. It is also much easier for transportation, delivery, and quality control using a generator produced isotope. The half-life of the parent isotope should be long while the half-life of the corresponding daughter isotope should be short. In addition, the cost for the production of the parent isotope and availability of the enriched source (for the production of the parent isotope) should also be considered.

¹⁸F is a cyclotron-produced PET isotope. The relatively long half-life (t_{1/2}=110 min) makes it possible for regional suppliers to ship ¹⁸F-FDG radiotracers to the clinical sites and for clinicians to collect useful images. ¹⁸F can be readily incorporated into endogenous biological compounds such as 2-deoxo-D-glucose. Following the foot-step of MRI, recent developments of mobile trailers for FDG PET imaging has made it possible for small institutions to have access to state-of-art PET services.

If the desired PET isotope is ¹⁸F, the target-specific PET radiopharmaceutical can be readily prepared according to the known procedures (Vaidyanathan, G. and Zalutsky, M. R. Bioconjugate Chem. 1990, 1, 269-273; Vaidyanathan, G. and Zalutsky, M. R. Nucl. Med. Biol. 1992, 19, 275-281; Vaidyanathan, G. and Zalutsky, M. R. Bioconjugate Chem. 1994, 5, 352-364; Vaidyanathan, G. and Zalutsky, M. R. Nucl. Med. Biol. 1995, 22, 759-764; Sutcliffe-Goulden et al. Bioorg. Med. Chem. Lett. 2000, 10, 1501-1503). In general, an active ¹⁸F-containing intermediate, such as N-succinimidyl 4-[¹⁸F]fluorobenzoate, is prepared in high yield and high radiochemical purity, and is then conjugated to an amino group of the SR-A receptor antagonist to form the 4-[¹⁸F]fluorobenzoyl conjugate. The ¹⁸F-labeled calpain and/or caspase inhibitor can be readily purified by simple filtration, by regular column chromatography, or by HPLC either using a size-exclusion or by reverse phase. The preferred procedure is that in which the ¹⁸F-labeled calpain, caspase inhibitor can be prepared in high specific activity and high radiochemical purity.

The radionuclide and the calpain or caspase inhibitor peptide, if the inhibitor comprises amino acid sequences, must be bound together. If the radionuclide is a radioactive halogen, the radioactive halogen may be bound directly to the peptide, such as by chemical reaction to a Tyr or Trp moiety of the peptide. If the radionuclide is a radioactive metal, the radioactive metal may be bound to the peptide by means of a chelating agent. A chelating group may be attached to the peptide by an amide bond or through a spacing group, as is known in the art.

Suitable chelating groups for chelating metal atoms are Nₜ S₍₄₋ₜ₎ tetradentate chelating agents, wherein t=2-4, or groups derived from ethylene diamine tetra-acetic acid (EDTA), diethylene triamine penta-acetic acid (DTPA), cyclohexyl 1,2-diamine tetra-acetic acid (CDTA), ethyleneglycol-0,0′-bis(2-aminoethyl)-N,N,N′,N′-tetra acetic acid (EGTA), N,N-bis(hydroxybenzyl)-ethylenediamine-N,N′-diacetic acid (HBED), triethylene tetramine hexa-acetic acid (TTHA), 1,4,7,10 tetraazacyclododecane-N,N′,N″,N′″-tetra-acetic acid (DOTA), hydroxyethyldiamine triacetic acid (HEDTA), 1,4,8,11-tetra-azacyclotetradecane-N,N′,N″,N′″-tetra-acetic acid (TETA), substituted DTPA, substituted EDTA.

As used herein, a “chelating group” is a group that can include a detectable radionuclide (e.g., a metallic radioisotope).

As used herein, a “detectable radionuclide” is any suitable radionuclide (i.e., radioisotope) useful in a diagnostic procedure in vivo or in vitro. Suitable detectable radionuclides include metallic radionuclides (i.e., metallic radioisotopes) and non-metallic radionuclides (i.e., non-metallic radioisotopes).

Suitable metallic radionuclides (i.e., metallic radioisotopes or metallic paramagnetic ions) include Antimony-124, Antimony-125, Arsenic-74, Barium-103, Barium-140, Beryllium-7, Bismuth-206, Bismuth-207, Cadmium-109, Cadmium-115m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-67, Erbium-169, Europium-152, Gallium-64, Gallium-68, Gadolinium-153, Gadolinium-157 Gold-195, Gold-199, Hafnium-175, Hafnium-175-181, Holmium-166, Indium-110, Indium-111, Iridium-192, Iron-55, Iron-59, Krypton-85, Lead-210, Manganese-54, Mercury-197, Mercury-203, Molybdenum-99, Neodymium-147, Neptunium-237, Nickel-63, Niobium-95, Osmium-185+191, Palladium-103, Platinum-195m, Praseodymium-143, Promethium-147, Protactinium-233, Radium-226, Rhenium-186, Rhenium-188, Rubidium-86, Ruthenium-103, Ruthenium-106, Scandium-44, Scandium-46, Selenium-75, Silver-110m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65, and Zirconium-95.

Preferably, the chelating group can include more than one metallic radioisotope. More preferably, the detectable chelating group can include 2 to about 10, 2 to about 8, 2 to about 6, or 2 to about 4 metallic radioisotopes.

Preferably, the non-metallic radionuclide can be a non-metallic paramagnetic atom (e.g., Fluorine-19); or a non-metallic positron emitting radionuclide (e.g., Carbon-11, Fluorine-18, Iodine-123, or Bromine-76).

Preferably, the compounds of the present invention can include more than one non-metallic radioisotope. More preferably, the compounds of the present invention can include 2 to about 10, 2 to about 8, 2 to about 6, or 2 to about 4 non-metallic radioisotopes. Examples of radiolabeled inhibitors, e.g. SJA-6017 and z-D-DCB, are shown below:

PET systems rely on an energy source that resides within a region of interest. To this end, positrons are positively charged electrons which are emitted by radionuclides that have been prepared using a cyclotron or other device. Radionuclides are employed as radioactive tracers called “radiopharmaceuticals” by incorporating them into substances such as glucose or carbon dioxide. While the radionuclides decay, the radionuclides emit positrons. The positrons travel a very short distance before they encounter an electron and, when the positron encounters an electron, the positron is annihilated and converted into two photons, or gamma rays. This annihilation event is characterized by two features which are pertinent to medical imaging and particularly to medical imaging using positron emission tomography (PET). First, each gamma ray has an energy of essentially 511 keV upon annihilation. Second, the two gamma rays are directed in substantially opposite directions.

In PET imaging, if the general locations of annihilations can be identified in three dimensions, a three dimensional image of a region of interest can be reconstructed for observation. To detect annihilation locations, a PET camera is employed. An exemplary PET camera includes a plurality of detectors and a processor which, among other things, includes coincidence detection circuitry. Each time an approximately 511 keV positron impacts a detector, the detector generates an electronic signal or pulse which is provided to the processor coincidence circuitry.

The coincidence circuitry identifies essentially simultaneous pulse pairs which correspond to detectors which are essentially on opposite sides of the imaging area. Thus, a simultaneous pulse pair indicates that an annihilation has occurred on a straight line between an associated pair of detectors. Over an acquisition period of a few minutes millions of coincidence events are recorded, each coincidence event is associated with a unique detector pair. After an acquisition period during which coincidence data is collected from every angle about an imaging area, recorded coincidence data can be used via any of several different well known procedures to construct images of radionuclide concentration in the region of interest. In the case of PET systems, PET data can be collected simultaneously from a volume within an object of interest so that a 3D image can be generated.

As is the case in virtually all imaging systems, one measure of the value of a PET system is throughput. To this end, in a radiology department the number of images generated is generally related to profitability with greater numbers of images translating into greater profitability. Thus, PET acquisition systems are generally designed to collect required imaging data rapidly. For this reason, one well accepted PET configuration is generally referred to as a full ring system which, as its label implies, includes a plurality of detector segments arranged to form an annular detector surface about an imaging area such that the system detects annihilation photons from many angles at a time. Hereinafter, for the purposes of this explanation a full ring detector system will be assumed unless indicated otherwise.

### Other Inhibitors

In another preferred embodiment, calpain inhibitors are identified based on the inhibition of enzymatic activity based on cell, tissue, and/or organ localization. For example, several typical tissue-specific calpains are known in vertebrates, including skeletal muscle p94 (nCL-1, calpain 3′, CAPN3), stomach nCL2 (CAPN4) and nCL 2′, and digestive tubule nCL4. Calpain inhibitors specific for tissue types can be determined based on the identity of each calpain. While p94 contains EF hands, it does not require calcium for proteinase activity. p94 has a domain IV sequence similar to that of μCL and mCL, but it does not bind to a small 30 kDa subunit (Kinbara et al. (1997) Arch. Biochem. Biophys. 342:99-107). p94 contains unique insertion sequences called IS1 and IS2, which are found in domain II and between domains III and IV, respectively. IS2 contains a nuclear-localization-signal-like basic sequence (Arg-Pro-Xaa-Lys-Lys-Lys-Lys-x-Lys-Pro). Connectin/titin binding is also attributed to IS2. p94 may change its localization in a cell-cycle dependent manner and may be involved in muscle differentiation by interacting with the MyoD family. In fact, a defect in the protease p94 is responsible for limb-girdle muscular dystrophy type 2A (LGMD2A). See Sorimachi et al. (1995) J. Biol. Chem. 270:31158-31162.

In another preferred embodiment, calpain inhibitors are identified based on the inhibition of specific substrate enzymatic activity. Calpains have broad physiological and pathological roles related to the enzymes' diverse population of substrates. Calpain substrates include “PEST” proteins, which have high proline, glutamine, serine, and threonine contents; calpain and calpastatin; signal transduction proteins including protein kinase C, transcription factors c-Jun, c-Fos, and α-subunit of heterotrimeric G proteins; proteins involved in cell proliferation and cancer including p53 tumor suppressor, growth factor receptors (e.g., epidermal growth factor receptor), c-Jun, c-Fos, and N-myc; proteins with established physiological roles in muscle including Ca²⁺-ATPase, Band III, troponin, tropomyosin, and myosin light chain kinase; myotonin protein kinase; proteins with established physiological roles in the brain and the central nervous system including myelin proteins, myelin basic protein (MBP), axonal neurofilament protein (NFP), myelin protein MAG; cytoskeletal and cell adhesion proteins including troponins, talin, neurofilaments, spectrin, microtubule associated protein MAP-2, tau, MAPIB, fodrin, desmin, α-actinin, vimentin, spectrin, integrin, cadherin, filamin, and N-CAM; enzymes including protein kinases A and C, and phospholipase C; and histones. See Sorimachi et al. (1997) J. Biochem. 328:721-732; Johnson et al. (1997) BioEssays 19(11): 1011-1018; Shields et al. (1999) J. Neuroscience Res. 55(5):533-541; and Belcastro et al. (1998) Mol. Cell. Biochem. 179 (1, 2): 135-145.

In another preferred embodiment, calpain inhibitors are identified based on classes of calpain substrates which include, but not limited to cytoskeletal and structural proteins, membrane bound receptors and proteins, calnodulin binding proteins, enzymes myofibrillar proteins and transcription factors. The examples of the first group include spectrin, MAP-2a, tau factor, neurofilament H, M and L, α-actinin. Examples of the second class include EGF receptor, AMPA-receptor, calcium pump, anion channel, calcium release channel, L-type calcium channel, G-proteins. Examples of the third class include calcium pump, calcineurin, CaM-dependent protein kinase II, myosin light chain kinase, neuromodulin, connexins, IP3 kinase. Examples of the fourth group include protein kinase C, HMG-CoA reductase, cAMP-dependent kinase, pyruvate kinase, phosphorylase kinase. Examples of the fifth group include troponin I, troponin T, tropomyosin, myosin. Examples of the sixth group include c-fos, c-jun, Pit-1, Oct-1, and b, c-Myc. See Wang, et al. (1997) Advances in Pharmacology, Volume 37.

Molecules from various chemical classes can be used as calpain inhibitors. As used herein, the term “calpain inhibitor” refers to those molecules which retard or inhibit the catalytic action of calpain. The various inhibitor tests can be carried out as follows:

Cathepsin B Test: Cathepsin B inhibition is determined by a method similar to that of S. Hasnain et al., J. Biol. Chem. 1993, 268, 235-240. For example, about 2 μl of an inhibitor solution is prepared from the chemical substance to be tested, a microbial or plant extract and DMSO (final concentration: 100 μM to 0.01 μM) are added to 88 μl of cathepsin B (from human liver supplied by Calbiochem, diluted to 5 units in 500 μM buffer). This mixture is preincubated at room temperature (25° C.) for 60 minutes, and then the reaction is started by adding 10 μl of 10 mM Z-Arg-Arg-pNA (in buffer with 10% DMSO). The reaction is followed at 405 nm in a microtiter plate reader for minutes. The IC₅₀ s are then determined from the maximum gradients.

Calpain I and II Test: The activity of the calpain inhibitors can be investigated in a colorimetric test using Hammarsten casein (Merck, Darmstadt) as substrate. The test is carried out in microtiter plates as published by Buroker-Kilgore and Wang in Anal. Biochem. 208, 1993, 387-392. The enzymes used are calpain I (0.04 U/test) from erythrocytes and calpain II (0.2 U/test) from kidneys, both from pigs, supplied by Calbiochem. The substances to be tested are incubated with the enzyme at room temperature for 60 minutes, the concentration of the solvent DMSO not exceeding 1%. After addition of the Bio-Rad color reagent, the optical density is measured at 595 μm in an SLT EAR 400 Easy Reader. The 50% enzyme activity is obtained from the optical densities determined at the maximum activity of the enzyme without inhibitors and the activity of the enzyme without addition of calcium.

The activity of calpain inhibitors can furthermore be determined using the substrate Suc-Leu Tyr-ACM. This fluorometric method is described by Zhaozhao Li et al., J. Med. Chem. 36 (1993), 3472-3480.

Since calpains are intracellular cysteine proteases, calpain inhibitors must pass through the cell membrane in order to prevent degradation of intracellular proteins by calpain. Some known calpain inhibitors, such as E 64 and leupeptin, cross cell membranes only poorly and, accordingly, show only a poor effect on cells, although they are good calpain inhibitors. It is therefore advantageous to carry out an additional test for the ability of potential calpain inhibitors to cross membranes, such as the human platelet test.

Platelet test to determine the cellular activity of calpain inhibitors: The calpain-mediated degradation of proteins in platelets is carried out as described by Zhaozhao Li et al., J. med. Chem. 36 (1993), 3472-3480. Human platelets are isolated from fresh sodium citrate blood from donors and adjusted to 10⁷ cells/ml in buffer (5 mM HEPES, 140 mM NaCl and 1 mg/ml BSA, pH 7.3).

Platelets (0.1 ml) are preincubated in 1 μl of various concentrations of potential inhibitors (dissolved in DMSO) for 5 minutes. This is followed by addition of the calcium ionophore A 23187 (1 μM) and calcium (5 mM) and further incubation at 37° C. for 5 minutes. After a centrifugation step, the platelets are taken up in SDS-PAGE sample buffer and boiled at 95° C. for 5 minutes, and the proteins are fractionated in an 8% gel. Degradation of the two proteins actin-binding protein (ABP) and talin is followed by quantitative densitometry. The half-maximum enzyme activity is determined with or, as control, without inhibitor from this. Also suitable for testing the ability to cross membranes are pieces of tissue such as brain sections or cell cultures.

The test for inhibition of calpain is carried out in cells which express this protein and/or in cells in which calpain specific substrates are present. Calpain types can be detected with specific antibody. If cells are stimulated with, for example, calcium and the appropriate ionophore, this leads to activation of calpain in the cell. Takaomi Saido described in J. Biochem. 11 (1992), 81-86 the autolytic transition of μ-calpain after activation, and detection with antibodies. Corresponding antibodies are produced for detecting substrate and cell selective calpain. Calpain inhibitors prevent the autolytic transition, and corresponding quantification is possible with antibodies.

Besides the in vitro tests described, just as the cellular platelet test, all other calpain tests known to the skilled worker are suitable, such as the test for inhibition of glutamate-induced cell death in cortical neurons (Maulucci-Gedde M. A. et al., J. Neurosci. 7, 1987: 357-368), calcium-mediated cell death in NT2 cells (Squier M. K. T. et al., J. Cell. Physiol., 35 159, 1994: 229-237, Patel T. et al., FASEB Journal 590, 1996: 587-597) or analysis of tissue samples for degradation products of proteins such as spectrin, MAP2 or Tau (Ami Arai et al., Brain Research, 1991, 555, 276-280, James Brorson et al., Stroke, 1995, 26, 1259-1267).

For in vitro tests on calpain or homologs thereof, cell target specific calpain is purified from tissues or cells in which the enzyme is expressed, such as the kidney, the thymus, the liver, the lung, or from cells or microorganisms which contain at least one gene copy and/or a vector with at least one gene copy of the desired calpain gene, and is used as crude extract or as pure enzyme.

For the methods according to the invention, the various calpain inhibitor tests are advantageously carried out in combination with the test for inhibition of cell-, tissue- and/or substrate-specific calpain enzyme activity by potential inhibitors. The inhibitors chosen for this inhibit either only the calpain type of interest and not the other calpains or, conversely, only the other calpains and not the calpain type of interest and at least one other calpain.

The various inhibitor tests are moreover carried out in such a way that, besides the test for the inhibitory effect of the test substance on each calpain type, calpain I and/or II, as a control the tests are carried out without the test substance. The inhibitory effects of the test substances can easily be detected by this test arrangement.

Another method according to the invention uses each calpain type for screening for new calpain inhibitors, it being possible for these inhibitors advantageously to inhibit all calpains in general or single calpains such as calpain I, II, and the like. The various test substances can for this purpose be tested singly or in parallel in test systems. The test substances are advantageously screened for their inhibitory effect in parallel automated test systems.

Any animal that expresses calpain activity can be used as a subject from which a biological sample is obtained in order to test for inhibition of calpain activity. Preferably, the subject is a mammal, such as for example, a human, dog, cat, horse, cow, pig, sheep, goat, chicken, primate, rat, or mouse. More preferably, the subject is a human. Particularly preferred are subjects suspected of having or at risk for developing traumatic or non-traumatic nervous system injuries, such as victims of brain injury caused by traumatic insults (e.g. gunshots wounds, automobile accidents, sports accidents, shaken baby syndrome), ischemic events (e.g. stroke, cerebral hemorrhage, cardiac arrest), spinal cord injury, neurodegenerative disorders (such as Alzheimer's, Huntington's, and Parkinson's diseases; Prion-related disease; other forms of dementia, and spinal cord degeneration), epilepsy, substance abuse (e.g., from amphetamines, methamphetamine/Speed, Ecstasy/MDMA, or ethanol and cocaine), and peripheral nervous system pathologies such as diabetic neuropathy, chemotherapy-induced neuropathy and neuropathic pain, peripheral nerve damage or atrophy (ALS), multiple sclerosis (MS).

### Administration of Labeled Inhibitors for Imaging

Another embodiment of the invention is the use of the protease inhibitor imaging agent to image neural damage in mammals, preferably humans. Neural imaging agents of the invention are administered to a mammal in need of such imaging, i.e., suspected of having neural damage, by intravenous injection. The imaging agent is administered in a single unit injectable dose at a concentration which is effective for diagnostic purposes. The imaging agent is administered intravenously in any conventional medium, such as isotonic saline, blood plasma, or biologically compatible isotonic buffers, such as phosphate, Hepes or Tyrode's buffer. Generally, the unit dose to be administered has a radioactivity of about 0.01 to about 100 mCi, preferably about 1 to 40 mCi. The solution amount to be injected as a unit dose is from about 0.1 ml to about 50.0 ml. Preferably, the amount injected is from about 0.5 to about 5 ml. Imaging of the central and peripheral nervous system can take place within a few minutes of injection. However, imaging can take place, if desired, several hours after injection. In most instances, a sufficient amount of the administered dose will accumulate in the desired area within a few minutes to a few hours after injection to permit the taking of scintigraphy images. This is an “effective diagnostic amount”. Any conventional method of scintigraphic imaging, planar, SPECT or PET, for diagnostic purposes, can be utilized in accordance with this invention.

Before conducting human studies, these protease inhibitor imaging agents can be first validated in animal model of traumatic brain injury and substance-abuse-induced brain injury (Table 2). After various time after injury event (e.g. 12 hours for TBI or 24 hours for Meth), the rats will be injected i.v. with calpain or caspase inhibitor-tracer (estimated 10 uCi, ˜1 nmol) under standard, non-restrained conditions. They will variously be sacrificed at 20, 40, 60, and 90 min., and brains will be sectioned via cryostat. Sections so obtained will then be exposed to film along with radiation activity standards, as described. We anticipate four-six rats in each group. Resultant autoradiograms will allow determination of the best time for highest signal to background noise, comparing the lesioned to non-lesioned brain regions, therefore validating the protease inhibitor imaging agents.

TABLE 2
Examples of established animal models of traumatic brain injury and substance-abuse
(methamphetamine/METH)-induced brain injury for the SEPCT/PET imaging studies

| Forms of Nervous | | |
|---|---|---|
| system injury | Rat model | References |
| Traumatic Brian injury | Control cortical Impact | Ringger, N. C., O'Steen, B. E., Brabham, |
| | device | J. G., Silver, X., Pineda, J., Wang, |
| | | K. K. W. and Hayes, R. L. (2005) A novel |
| | | marker for traumatic brain injury: CSF |
| | | alphaII-spectrin breakdown product |
| | | levels. J. Neurotrauma 21, 1443-1456. |
| Substance exposure | METH: i.p. injection 10 mg/ | Warren, M. W., Kobeissy, F. H., Hayes, |
| induced brain injury | kg × 3-4 doses in 1 h interval | R. L., Gold, M. S., Wang, K. K. W (2005) |
| | | Concurrent calpain and caspase-3 |
| | | mediated proteolysis of alphaII-spectrin |
| | | and tau in rat brain after |
| | | methamphetamine exposure: A similar |
| | | profile to traumatic brain injury. Life |
| | | Sciences 78: 301-309. |

### Kits

Still another embodiment of the invention is a kit for the preparation of the neural imaging agents. The kit includes a carrier for holding the kit components and containers of the neural imaging agent, reducing agent and buffer.

In another preferred embodiment, a kit comprises a calpain and/or caspase inhibitor and a radionuclide; wherein, the calpain inhibitor comprises calpain inhibitor I, calpain inhibitor II, N-acetyl-Leu-Leu-norleucinal, N-acetyl-Leu-Leu-methioninal, calpeptin, E-64, E-64-c, E-64-d, Z-VF-CHO, Z-Leu-Leu-CHO, leupeptin (N-acetyl-Leu-Leu-argininal), oxoamide inhibitor molecules AK295, AK275, MDL28170 CX275, SJA6017, SNJJ-1715 or SNJ-1945; and, the caspase inhibitor comprises Z-D-DCB (Z-Asp-CH2OC(O)-2,6-dichlorobenzene or zAsp-CH2-DCB), Z-VAD-DCB, zVADfmk, acetyl-DEVD-CHO, DEVD-fluoromethylketone, Z-Val-DL-Asp-fluoromethylketone, Z-Val-DL-Asp(OMe)-fluoromethylketone M826 or IDN-6556. Preferably the radionuclide comprises ¹¹C, ¹⁴C, ³H, ¹⁸F, ^{99m}T, ¹⁸⁶Re, ¹⁸⁸Re, ¹¹¹In, ⁶⁷Ga, ⁶⁸Ga, ²⁰¹Tl, ⁵²Fe, ²⁰³Pb, ⁵⁸Co, ⁶⁴Cu, ¹²³I, ¹²⁴I, ¹²⁵I, ¹³¹I, ²¹⁰At, ⁷⁶Br, ⁷⁷Br, ¹⁸F, ³²P, ³⁵S, ¹²³I, ¹²⁵I, or ¹³¹I.

The labeled inhibitors may be combined with carrier materials such as saline, and adjuvants, such as acids or bases added to alter the pH and/or act as buffers and/or preservatives. The use of carriers and adjuvants is well known to those skilled in the art.

The invention may be provided to the user by providing a suitable radiolabeled inhibitor of the invention in a carrier, with or without adjuvants, or by providing some or all of the necessary components in a kit. The use of a kit is particularly convenient since some of the components have a limited shelf life, particularly when combined. A suitable kit may include one or more of the following components (i) an inhibitor, (ii) a chelating agent, (iii) a carrier solution, (iv) a radioisotope, (v) a reducing agent, and (vi) instructions for their combination. Depending on the form of the radionuclide, a reducing agent may be a necessary to prepare the radionuclide for reaction with the inhibitor. Suitable reducing agents include Ce (III), Fe (II), Cu (I), Ti (III), Sb (III), and Sn (II).

For reasons of stability, it is generally preferred that the inhibitor be in a dry, lyophilized condition. The user adds the carrier solution to the dry inhibitor to reconstitute it. If it is desired to provide the inhibitor in solution form, it may be necessary to store it at lower temperatures than the dry form.

For diagnosis of neural injury or damage, the radiolabeled compounds are administered in an amount effective to allow neural imaging. The selection of the effective amount is within the skill of one skilled in the art.

In the methods of the present invention, the compounds may be administered orally, parenterally, by inhalation spray, topically, rectally, nasally, buccally, vaginally or via an implanted reservoir in dosage formulations containing conventional non-toxic pharmaceutically-acceptable carriers, adjuvants and vehicles. The term parenteral as used herein includes subcutaneous, intravenous, intramuscular, intraperitoneal, intrathecal, intraventricular, intrasternal or intracranial injection and infusion techniques. Invasive techniques are preferred, particularly direct administration to damaged neuronal tissue.

The compounds may also be administered in the form of sterile injectable preparations, for example, as sterile injectable aqueous or oleaginous suspensions. These suspensions can be formulated according to techniques known in the art using suitable dispersing or wetting agents and suspending agents. The sterile injectable preparations may also be sterile injectable solutions or suspensions in non-toxic parenterally-acceptable diluents or solvents, for example, as solutions in 1,3-butanediol. Among the acceptable vehicles and solvents that may be employed are water, Ringer's solution and isotonic sodium chloride solution. In addition, sterile fixed oils are conventionally employed as solvents or suspending mediums. For this purpose, any bland fixed oil such as a synthetic mono- or di-glyceride may be employed. Fatty acids such as oleic acid and its glyceride derivatives, including olive oil and castor oil, especially in their polyoxyethylated forms, are useful in the preparation of injectables. These oil solutions or suspensions may also contain long-chain alcohol diluents or dispersants.

Additionally, the compounds may be administered orally in the form of capsules, tablets, aqueous suspensions or solutions. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. The oral dosage forms may further contain sweetening and/or flavoring and/or coloring agents.

The compounds may further be administered rectally in the form of suppositories. These compositions can be prepared by mixing the drug with suitable non-irritating excipients which are solid at room temperature, but liquid at rectal temperature such that they will melt in the rectum to release the drug. Such excipients include cocoa butter, beeswax and polyethylene glycols.

Moreover, the compounds may be administered topically, especially when the conditions addressed for treatment involve areas or organs readily accessible by topical application, including neurological disorders of the eye, the skin or the lower intestinal tract.

For topical application to the eye, or ophthalmic use, the compounds can be formulated as micronized suspensions in isotonic, pH adjusted sterile saline or, preferably, as a solution in isotonic, pH adjusted sterile saline, either with or without a preservative such as benzylalkonium chloride. Alternatively, the compounds may be formulated into ointments, such as petrolatum.

For topical application to the skin, the compounds can be formulated into suitable ointments containing the compounds suspended or dissolved in, for example, mixtures with one or more of the following: mineral oil, liquid petrolatum, white petrolatum, propylene glycol, polyoxyethylene polyoxypropylene compound, emulsifying wax and water. Alternatively, the compounds can be formulated into suitable lotions or creams containing the active compound suspended or dissolved in, for example, a mixture of one or more of the following: mineral oil, sorbitan monostearate, polysorbate 60, cetyl ester wax, cetearyl alcohol, 2-octyldodecanol, benzyl alcohol and water.

Topical application to the lower intestinal tract can be effected in rectal suppository formulations (see above) or in suitable enema formulations.

The compounds of the present invention may be administered by a single dose, multiple discrete doses or continuous infusion. Since the compounds are small, easily diffusible and relatively stable, they are well suited to continuous infusion. Pump means, particularly subcutaneous pump means, are preferred for continuous infusion.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### EXAMPLES

### Example 1

### Pro-Drug Synthesis

To enhance the brain residency of the selected “inhibitor-ligands,” a pro-drug strategy that promotes the “trapping” of their precursors in the CNS are evaluated (Prokai et al., 2000). Specifically, the short (ca. 11-min) in vivo half-life of SJA-6017 after intravenous administration represents a serious problem, when its potential to yield a useful CNS-diagnostic agent upon making it “PET-visible” is considered. (Although SJA-6017 is expected to cross the BBB due to its adequate lipophilicity characterized by the calculated log P of 2.48 (Ghose et al., 1988), it will also rapidly efflux from the CNS as its blood concentration declines). Pharmacokinetic evaluation of Z-D-DCB (calc. log P=4.15) has not been completed; however, a pro-drug approach similar to that employed to SJA-6017 will also be beneficial for the prospective diagnostic application. By definition, pro-drugs are inactive derivatives (i.e., they do bind to the cognate receptors) that convert to a pharmacologically active species in vivo. The following scheme outlines the method employed to the selected inhibitor-ligands (see FIG. 5).

N-Methyl-3-carbonyl-1,4-dihydropyridinyl will be used as “promoiety” (pmty)⁽¹⁾ for SJA-6017. The attachment of this group will yield a pro-drug whose calculated log P (1.94) is only slightly different from the parent compound; hence, it should readily distribute into the CNS. A rapid metabolic conversion (oxidation) of the non-ionic pmty⁽¹⁾ to the corresponding membrane-impermeable pyridinium ion as a CNS-trapped intermediate before the (preferably slow) dissociatior of the oxidized promoiety from SJA-6017 is the process responsible for the increase in the residence time of the drug in the CNS. The oxidative conversion of a dihydropyridine to pyridinium occurs ubiquitously, and it is analogous to the oxidation of NAD(P)H, a coenzyme associated with many oxidoreductases and cellular respiration. Any of the oxidized form in the periphery will, however, be rapidly eliminated, as it is polar and an excellent candidate for elimination by the kidney and bile. Thus, concentration of the active drug will remain low in the periphery, which is beneficial for the intended diagnostic purposes. The attachment pmty⁽¹⁾ to SJA-6017 will be accomplished first by reacting the parent compound with 1 molar equivalent (eq) of nicotinic anhydride at room temperature in dry pyridine (Brewster et al., 1991). The nicotinoylated compound will then be reacted with excess methyl iodide (at room temperature in acetone as a solvent) followed by reduction with sodium dithionite (at room temperature in methanol as a solvent). The synthesis of the pro-drug for Z-D-DCB will start by the esterification (in dimethylformamide as a solvent) of the side-chain COOH of the Asp residue with 3-pyridinecarboxylic acid N-hydroxymethylamide and in the presence of N-cyclohexylcarbodiimide, N′-methyl polystyrene and N-(methylpolystyrene)-4-(methylamino)pyridine (Prokai-Tatrai et al., accepted). N-methylation and dithionite reduction to yield the pro-drug with pmty⁽²⁾ will be done similarly to those employed in the synthesis of the pmty⁽¹⁾-SJA-6017 conjugate (calc. log P=3.15). High-performance liquid chromatography will be used to measure the compounds in question in the exploratory phase. New pro-drugs by systematic modification of the promoieties are also designed.

### Example 2

### Validation of Pro-Drug Activity in Culture Cells

The synthesized promoiety-conjugates Compound 1 and Compound 2, will be tested for oxidation stability at 37° C. in buffer (pH 7.4), rat plasma and rat-brain homogenate to probe the kinetics of the expected conversion to the pyridinium-ion intermediates. We expect a rapid oxidation followed by a slow(er) hydrolysis in biological media, which is ideal to provide a sustained, “quasi” steady-state level of SJA-6017 or Z-D-DCB in the brain for at least an hour. Compound 1 for calpain and Compound 2 will be further tested in in situ calpain and caspase inhibition analysis in cell culture. This may be important since both proteases are intracellular, so for the in vivo uses of the inhibitor-ligands, compounds must have the ability to penetrate cells and retain potency while inside cells. We will use neuroblastoma cell line SH-SY5Y, which can be manipulated to produce calpain-activation and necrosis with a neurotoxin maitotoxin (0.3 nM, Calbiochem) or caspase activation and apoptosis with calcium-chelator EGTA (5 mM) as we have shown before (McGinnis, Wang, Gnegy, (1999) J. Neurochem. 72, 1853-1863). Importantly the protease-produced spectrin breakdown products SBDP150 and SBDP145 (with maitotoxin) and SBDP150 and SBDP120 (with EGTA) are readily produced and quantitatively detected by Western blot analysis, which parallel the formation of SBDP's in brain injury in vivo. Prototype calpain inhibitor SJA6017 blocked SBDP145 at IC₅₀ of 0.2 μM, while caspase inhibitor z-D-DCB blocks SBDP120 at IC₅₀ of 5 μM. Our modified compounds 1 and 2 will be titrated and pretreated cells 1 h prior to being challenged with either maitotoxin or EGTA to assess their in situ potency as well as selectivity.

### Example 3

### Ex Vivo Autoradiography with Calpain and Caspase Inhibitor-Ligands

These experiments will employ ex vivo autoradiography, to examine binding distributions and kinetics of the inhibitor-ligands in rat brain in situ. Two injury paradigms (TBI and Meth exposure) will be used. Adjacent tissue sections will be incubated within six concentrations (0.5, 1, 5, 10, 50 and 100 nM) of one of the two inhibitor-ligands. To provide adequate tissue and statistics, tissue from six rats for each group will be needed, thus a total of 24 rats will be needed. Based on our existing experience of the models and the general technology employed, we are confident that the experiments can be accomplished on schedule (3 months). The experimental details are as follows:

(A) For TBI, on Day 1, large, young male S-D rats, (400-500 gm; from Charles Rivers labs) will be placed under inhalational anesthesia (isoflourane) and receive a unilateral cortical impact lesion (1.6 mm force) to the right hemisphere. They are then allowed to recover under standard procedures.

(B) For Meth neurotoxicity, on Day 1 rats were intraperitoneally (i.p.) injected with a dose of Meth or saline in a bolus of 0.3 cc after a brief anaesthetization (isoflourane). Our pilot data suggesting that both 20 mg/kg i.p. dose and 24 h time points are optimal in producing protease activation signals in the cerebral cortex and hippocampus. Twelve hours (TBI) or 24 hours later (Meth)) rats will be sacrificed with i.p. pentobarbital. Following sacrifice, brains are immediately removed and frozen in dry ice slurry, then sectioned via cryostat (20 μm) onto. SuperFrost Plus Gold® (Fisher Scientific) slides and frozen at −80° C. until use. After incubation in Tris-buffered saline (pH 7.4) to remove endogenous ligands, sections are incubated with similarly buffered radioligands (concentrations, below) in great excess (>4 ml per slide) for 90 min. then rinsed in 4° C. buffer, followed by 4° C. deionized water and dried quickly in a stream of cool, dry air. Liquid scintillation counting of radioactivity in a 100 μl aliquot of incubation medium at the end of experiments measures radioligand concentration at equilibrium. Slides are then apposed to X-ray film, together with ¹⁴C radiation activity standards for appropriate exposure times (estimated at 2-4 weeks). The resultant autoradiographs are analyzed for regional photographic optical densities as previously described (Baxter L R, Ackermann R F, Clark E C, Baxter J E: Brain Behav Evol. 2001; 57:169-184; Baxter L R, Clark E C, Ackermann R F, Lacan G, Melega W P: Brain Behav Evol. 2001a; 57:185-201; Clark E C, Baxter L R: Mammal-like striatal functions in Anolis. I. Brain Behav Evol. 2000; 56:235-248; Clark E C, Baxter L R, Dure L S, Ackermann R F, Kemp G F, Bachus S E. Brain Behav Evol. 2000; 56:249-258) to determine regional radioligand retention. (The ratio of photographic density to radiation exposure, as calculated via the applied ¹⁴C standards, is highly log/log-linear within the exposures used with the film used (r≧0.98). Calculations of concentrations of free vs. tissue-bound radioligand are then used for Scatchard plot analyses to estimate Kd of this ligand for receptor types, and give an estimate of B-max. This is done via incubating adjacent tissue samples in varying concentrations (0.5, 1, 5, 10, 50 and 100 nM) of one of the two inhibitor-ligands. Results of that will be used to estimate the appropriate concentration range for Scatchard analysis, as our estimates of Kd now range up to 40 nM. Results at 10 nM (e.g. time to adequate exposure) will determine whether to target a range for a higher or lower Kd.

### Example 4

### In Vivo Labeling of Injured Brain Regions with Inhibitor-Ligands

Before conducting human studies, these protease inhibitor imaging agents can be first validated in animal model of traumatic brain injury and substance-abuse-induced brain injury (Table 2). After various time after injury event (e.g. 12 hours for TBI or 24 hours for Meth), the rats will be injected i.v. with calpain or caspase inhibitor-tracer (estimated 10 μCi, ˜1 nmol) under standard, non-restrained conditions. They will variously be sacrificed at 20, 40, 60, and 90 min, and brains will be sectioned via cryostat. Sections so obtained will then be exposed to film along with radiation activity standards, as described. We anticipate four-six rats in each group. Resultant autoradiograms will allow determination of the best time for highest signal to background noise, comparing the lesioned to non-lesioned brain regions, therefore validating the protease inhibitor imaging agents.

In this setting, we will explore the potential of the calpain and caspase inhibitor-ligand (or a modification thereof, if Experiment 1 dictates) as an in vivo radioligand that might be suitable for use with SPECT, microPET and PET in the future. Animals will be again subjected to two modes of brain injury (TBI and Meth exposure), one of the two inhibitor-ligand will be injected into the animals. They will be sacrificed at 4 different time points (20, 40, 60, and 90 min). Six animals will be used for each group for statistical analysis. Thus, a total of 48 rats are needed. The experimental details are as follows:

For (A) TBI, rats will undergo the same anesthesia and brain lesions, and also have standard stylistic jugular or femoral catheters implanted. For (B) Meth neurotoxicity, rats will undergo the same anesthesia and Meth administration, and also have standard sylastic juglar or femeral catheters implanted.

After 12 hours (for TBI) or 24 hours (for Meth), the rats will be injected i.v. with calpain or caspase inhibitor-tracer (estimated 10 μCi, ˜1 nmol) under standard, non-restrained conditions. They will be sacrificed at 20, 40, 60, and 90 min, and brains rendered via cryostat, as previously described. Sections so obtained will then be exposed to film along with radiation activity standards, as described. We anticipate four-six rats in each group.

Resultant autoradiograms will allow determination of the best time for highest signal to background noise, comparing the lesioned to non-lesioned brain regions.

Additional rats are to be sacrificed at the optimal image time, but with small blood samples taken via femeral artery at appropriate time points. These will be measured for specific radio-activity (via scintillation counter) to obtain a time curve for a formal tracer-kinetic, three compartment analyses. Given where the compounds will be labeled, metabolism will not produce a binding radioactive metabolite, so HPLC can be used to simply determine the % radioactivity that is active ligand, vs. inactive over time for the model.

### Example 5

### Administration of Labeled Inhibitors for Imagining Animals and in Human

Another embodiment of the invention is the use of the protease inhibitor imaging agent to image neural damage in mammals, preferably humans. Neural imaging agents of the invention are administered to a mammal in need of such imaging, i.e., suspected of having neural damage, by intravenous injection. The imaging agent is administered in a single unit injectable dose at a concentration which is effective for diagnostic purposes. The imaging agent is administered intravenously in any conventional medium, such as isotonic saline, blood plasma, or biologically compatible isotonic buffers, such as phosphate, Hepes or Tyrode's buffer. Generally, the unit dose to be administered has a radioactivity of about 0.01 to about 100 mCi, preferably about 1 to 40 mCi. The solution amount to be injected as a unit dose is from about 0.1 ml to about 50.0 ml. Preferably, the amount injected is from about 0.5 to about 5 ml. Imaging of the central and peripheral nervous system can take place within a few minutes of injection. However, imaging can take place, if desired, several hours after injection. In most instances, a sufficient amount of the administered dose will accumulate in the desired area within a few minutes to a few hours after injection to permit the taking of scintigraphy images. This is an “effective diagnostic amount”. Any conventional method of scintigraphic imaging, planar, SPECT or PET, for diagnostic purposes, can be utilized in accordance with this invention.

### OTHER EMBODIMENTS

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

1. A composition comprising a calpain and/or caspase inhibitor covalently bonded or ligated to a radionuclide wherein the calpain inhibitor comprises calpain inhibitor I, calpain inhibitor II, N-acetyl-Leu-Leu-norleucinal, N-acetyl-Leu-Leu-methioninal, calpeptin, E-64, E-64-c, E-64-d, Z-VF-CHO, Z-Leu-Leu-CHO, leupeptin (N-acetyl-Leu-Leu-argininal), oxoamide inhibitor molecules AK295, AK275, MDL28170 CX275, SJA6017, SNJJ-1715 or SNJ-1945; the caspase inhibitor inhibits activity of caspase-1, caspase-2, caspase-3, caspase-4, caspase-5, caspase-6, caspase-6, caspase-7, caspase-8, or caspase-9 or caspase-12 and comprises Z-D-DCB (Z-Asp-CH2OC(O)-2,6-dichlorobenzene or zAsp-CH2-DCB), Z-VAD-DCB, zVADfmk, acetyl-DEVD-CHO, DEVD-fluoromethylketone, Z-Val-DL-Asp-fluoromethylketone, Z-Val-DL-Asp(OMe)-fluoromethylketone M826 or IDN-6556, and; the radionuclide comprises ¹¹C, ¹⁴C, ³H, ¹⁸F, ^{99m}T, ¹⁸⁶Re, ¹⁸⁸Re, ¹¹¹In, ⁶⁷Ga, ⁶⁸Ga, ²⁰¹Tl, ⁵²Fe, ²⁰³Pb, ⁵⁸Co, ⁶⁴Cu, ¹²³I, ¹²⁴I, ¹²⁵I, ¹³¹I, ²¹⁰At, ⁷⁶Br, ⁷⁷Br, ¹⁸F ³²P, ³⁵S, ¹²³I, ¹²⁵I, or ¹³¹I.

2-7. (canceled)

8. A composition comprising a cathepsin, metalloprotease, proteasome, granzyme B, metalloproteinase inhibitor, or other protease inhibitor and a radionuclide wherein the cathepsin inhibitor comprises CA-074, CA-074-Me, CP-1, CP-2, Napsul-Ile-Trp-CHO, and Pepstatin A; the Granzyme B inhibitor comprises 3,4-Dichloroisocoumarin; the metalloproteinase inhibitor comprises Actinonin, CL-82198, Epigallocatechin gallate, GM6001, NNGH (BML-205), BB-94 or KB-R7785; the proteasome inhibitor comprises Lactacystin, Clasto-Lactacystin 13-lactone, Epoxomicin, Gliotoxin, MG-132, MG-262, PS-341; Z-Ile-Glu(OtBu)-Ala-Leu-CHO (IGAL), and MN-519; the other protease inhibitor comprises AAF-CMK, Arphamenine A, Bestatin (Ubenimex), Boc-GVV-CHO; Captopril, Elastatinal, Phosphoramidon, PPACK, Z-Prolyl-Prolinal, Thiorphan (DL), TLCK, and TPCK; and, the radionuclide comprises ¹¹C, ¹⁴C, ³H, ¹⁸F, ^{99m}T, ¹⁸⁶Re, ¹⁸⁸Re, ¹¹¹In, ⁶⁷Ga, ⁶⁸Ga, ²⁰¹Tl, ⁵²Fe, ²⁰³Pb, ⁵⁸Co, ⁶⁴Cu, ¹²³I, ¹²⁴I, ¹²⁵I, ¹³¹I, ²¹⁰At, ⁷⁶Br, ⁷⁷Br, ¹⁸F, ³²P, ³⁵S, ¹²³I, ¹²⁵I, or ¹³¹I.

9-18. (canceled)

19. A method of neural imaging in a patient or animal comprising:
administering to a patient a neural injury specific imaging agent, wherein the neural injury specific imaging agent comprises a calpain and/or caspase inhibitor and a radionuclide wherein the radionuclide comprises ¹¹C, ¹⁴C, ³H, ¹⁸F, ^{99m}T, ¹⁸⁶Re, ¹⁸⁸Re, ¹¹¹In, ⁶⁷Ga, ⁶⁸Ga, ²⁰¹Tl, ⁵²Fe, ²⁰³Pb, ⁵⁸Co, ⁶⁴Cu, ¹²³I, ¹²⁴I, ¹²⁵I, ¹³¹I, ²¹⁰At, ⁷⁶Br, ⁷⁷Br, ¹⁸F, ³²P, ³⁵S, ¹²³I, ¹²⁵I, or ¹³¹I;
detecting a radiolabeled calpain and/or caspase inhibitor; wherein the neural injury comprises any one of: damage to the nervous system, including retinal ganglion cells; a traumatic brain injury; a stroke related injury; cerebral ischemia, shaken baby syndrome, a cerebral aneurism related injury; demyelinating diseases; a spinal cord injury, including monoplegia, diplegia, paraplegia, hemiplegia and quadriplegia; a neuroproliferative disorder or neuropathic pain syndrome; stroke, concussion, post-concussion syndrome, cerebral ischemia, neurodegenerative diseases brain injuries, infection or neuropathies;
detecting the neural injury specific imaging agent positron emission tomography (PET), single photon emission computed tomography (SPECT), radioscintigraphy, magnetic resonance imaging (MRI), computed tomography (CT scan); and,
imaging neural damage in the patient or animal.

20. A method of neural imaging in a patient or animal comprising:
administering to a patient a neural injury specific imaging agent, wherein the neural injury specific imaging agent comprises a calpain and/or caspase inhibitor and a radionuclide; wherein the radionuclide comprises ¹¹C, ¹⁴C, ³H, ¹⁸F, ^{99m}T, ¹⁸⁶Re, ¹⁸⁸Re, ¹¹¹In, ⁶⁷Ga, ⁶⁸Ga, ²⁰¹Tl, ⁵²Fe, ²⁰³Pb, ⁵⁸Co, ⁶⁴Cu, ₁₂₃I, ¹²⁴I, ¹²⁵I, ¹³¹I, ²¹⁰At, ⁷⁶Br, ⁷⁷Br, ¹⁸F, ³²P, ³⁵S, ¹²³I, ¹²⁵I, or ¹³¹I;
detecting a radiolabeled cathepsin, metalloprotease, proteasome, granzyme B, or other protease inhibitor; wherein the neural injury comprises any one of: damage to the nervous system, including retinal ganglion cells; a traumatic brain injury a stroke related injury, cerebral ischemia, shaken baby syndrome, a cerebral aneurism related injury; demyelinating diseases; a spinal cord injury, including monoplegia, diplegia, paraplegia, hemiplegia and quadriplegia; a neuroproliferative disorder or neuropathic pain syndrome; stroke, concussion, post-concussion syndrome, cerebral ischemia, neurodegenerative diseases brain injuries, infection or neuropathies; and,
imaging neural damage in the patient or animal.

21-24. (canceled)

25. The method of claim 20, wherein the neural injury specific imaging agent is detected by positron emission tomography (PET), single photon emission computed tomography (SPECT), radioscintigraphy, magnetic resonance imaging (MRI), computed tomography (CT scan).

26. A method of organ or multi-organ injury imaging in a patient or animal comprising:
administering to a patient a organ injury specific imaging agent wherein the organ injury or multi-organ injury specific imaging agent comprises a calpain and/or caspase inhibitor or a cathepsin, metalloprotease, proteasome, granzyme B, or other protease inhibitor and a radionuclide, wherein the radionuclide is ¹¹C, ¹⁴C, ³H, ¹⁸F, ^{99m}T, ¹⁸⁶Re, ¹⁸⁸Re, ¹¹¹In, ⁶⁷Ga, ⁶⁸Ga, ²⁰¹Tl, ⁵²Fe, ²⁰³Pb, ⁵⁸Co, ⁶⁴Cu, ¹²³I, ¹²⁴I, ¹²⁵I, ¹³¹I, ²¹⁰At, ⁷⁶Br, ⁷⁷Br, ¹⁸F, ³²P, ³⁵S, ¹²³I, ¹²⁵I, or ¹³¹I; and,
detecting radiolabeled calpain and/or caspase inhibitor; thereby imaging organ damage in the patient or animal.

27. The method of claim 26, wherein the organ injury comprises any damage, injury or infection, functional failure to specific organs such as liver, kidney, prostate, lung, skeletal muscle, heart, pancreas, stomach, small and large intestine, bladder and the reproductive system functional failure to multi-organs, trauma-hemorrhagic shock and sepsis.

28-31. (canceled)

32. The method of claim 26, wherein the organ specific imaging agent is detected by positron emission tomography (PET), single photon emission computed tomography (SPECT), radioscintigraphy, magnetic resonance imaging (MRI), computed tomography (CT scan).

33. The composition of claim 26, wherein the calpain inhibitor comprises calpain inhibitor I, calpain inhibitor II, N-acetyl-Leu-Leu-norleucinal, N-acetyl-Leu-Leu-methioninal, calpeptin, E-64, E-64-c, E-64-d, Z-VF-CHO, Z-Leu-Leu-CHO, leupeptin (N-acetyl-Leu-Leu-argininal), oxoamide inhibitor molecules AK295, AK275, MDL28170 CX275, SJA6017, SNJ-1715 or SNJ-1945.

34. The composition of claim 26, wherein the caspase inhibitor inhibits activity of caspase-1, caspase-2, caspase-3, caspase-4, caspase-5, caspase-6, caspase-6, caspase-7, caspase-8, or caspase-9 or caspase-12.

35. The composition of claim 26, wherein the caspase inhibitor comprises Z-D-DCB (Z-Asp-CH2OC(O)-2,6-dichlorobenzene or zAsp-CH2-DCB), Z-VAD-DCB, zVADfmk, acetyl-DEVD-CHO, DEVD-fluoromethylketone, Z-Val-DL-Asp-fluoromethylketone, Z-Val-DL-Asp(OMe)-fluoromethylketone M826 or IDN-6556.

36-37. (canceled)
